(19) United States
(12) Patent Application Publication
(10) Pub. No.: US 2022/0233125 A1
(43) Pub. Date: Jul. 28, 2022

(54) INTRAOPERATIVE 'NON-LIFTING' PERIPHERAL NERVE ACTION POTENTIAL RECORDING

(71) Applicant: THE JOHNS HOPKINS UNIVERSITY, Baltimore, MD (US)

(72) Inventors: Matthias Ringkamp, Baltimore, MD (US); Gang Wu, Princeton, NJ (US); Gene Yevgeny Fridman, Baltimore, MD (US)

(21) Appl. No.: 17/617,233

(22) PCT Filed: Jun. 8, 2020
(86) PCT No.: PCT/US2020/036599

§ 371 Date: Dec. 7, 2021

Related U.S. Application Data
(60) Provisional application No. 62/858,418, filed on Jun. 7, 2019.

(51) Int. Cl. A61B 5/294; A61B 5/296; A61B 5/395; A61N 1/36; A61N 1/04; A61N 1/05

(52) U.S. Cl. CPC A61B 5/294; A61B 5/296; A61B 5/395; A61N 1/36014; A61N 1/0408; A61N 1/0476; A61N 1/0558; A61N 1/3605

(57) ABSTRACT

The present invention is directed to an electrode system for recording nerve action potential (NAP) from surgically exposed nerve and methods for using such an electrode system. Electrophysiological methods are used during repair surgery of peripheral nerve trauma (PNT). PNT is a major medical problem with an annual incidence similar to that of epilepsy. Surgical intervention is provided based on the severity of nerve injury which is determined preoperatively and intraoperatively mainly by electrophysiological assessments. Among those, intraoperative nerve action potential (NAP) or compound action potential (CNAP) recording is preferred for direct assessment of functional continuity of the nerve.

### CROSS REFERENCE TO RELATED APPLICATIONS

This application claims the benefit of U.S. Provisional Patent Application No. 62/858,418 filed on Jun. 7, 2019, which is incorporated by reference, herein, in its entirety.

### GOVERNMENT SUPPORT

This invention was made with government support under AR070875 awarded by the National Institutes of Health. The government has certain rights in the invention.

### FIELD OF THE INVENTION

The present invention relates generally to medical devices. More particularly, the present invention relates to intraoperative non-lifting peripheral nerve action potential recording.

### BACKGROUND OF THE INVENTION

Intraoperative nerve action potential (NAP) or compound nerve action potential (CNAP) recording is a proven useful tool to guide surgeon's decisions about surgical approaches during various nerve repairs. Using this tool in combination with other assessments, the surgical team can determine the exact location of a nerve lesion. Clinical data from retrospective studies have shown that the surgical outcome is improved by utilizing this tool to select the repair method.

Intraoperative NAP recording can be technically challenging although the current methods are simplified by the use of handheld electrodes applied to surgically exposed nerves. The typical recommendation is to lift the nerve out of the surgical field during testing. After dissection and isolation, a surgically exposed nerve is typically lifted by two pairs of electrodes (one for recording and another for stimulation) and the nerve segment between the two electrodes is often suspended with no direct contact with tissue underneath. The main limitations noted are the length of the exposed nerve segment and the presence of a large stimulus artifact. Despite these major challenges, no fundamental understanding and/or improvement of the methods have been reported. Despite considerable improvements in hardware and software employed for neurophysiological recordings, the new generation of IONM (intraoperative neurophysiological monitoring) machines do not resolve the existing technical challenges of NAP recordings.

Accordingly, there is a need in the art for a device and method for intraoperative non-lifting peripheral nerve action potential recording.

### SUMMARY OF THE INVENTION

The foregoing needs are met, to a great extent, by the present invention which provides a system and method including an electrode for stimulation or recording of a compound nerve action potential from surgically exposure peripheral nerve. The electrode includes a number of insulated metal prongs, such that the nerve can be sandwiched between the prongs, without lifting the nerve. The prong has an area for contact for nerve stimulation or recording. The electrode also includes an elastic base or body, configured to provide the nerve with a predetermined tension when the nerve is sandwiched by the prongs.

In accordance with an aspect of the present invention, the prongs include insulation that covers all of the prong- except for the area for contact for nerve stimulation or recording. The base is formed from a plastic or silicone and has elastic properties. In another configuration when a clamp body is used, the body is formed from a silicone and has elastic properties, In addition, the body includes two elastic clips to be used to open and close the electrode. The prongs include a metal core.

In accordance with an aspect of the present invention, an elastic base has at least three projections. At least one of the at least three projections is on a top side of a nerve and at least two of the at least three projections are on a bottom side of the nerve. The nerve is sandwiched by the at least three projections. The system includes an electrode for stimulation or recording of a nerve action potential comprising at least two metal contacts. The elastic base and electrode are self-insulating and self-holding, such that a non-lifting nerve action potential can be stimulated and/or recorded.

In accordance with another aspect of the present invention, the middle prong and the two outer prongs include insulation that covers all of the middle prong and the two outer prongs except for an area of the metal contact for nerve stimulation or recording. The base is formed from a plastic or silicone. A pressure is applied to the nerve, because it is sandwiched by the at least three projections. The pressure is less than what would be damaging to the nerve for a duration of a test of the compound nerve action potential. The at least three projections can include grooves at a location of the electrode contact with the nerve, such that the nerve is self-positioned on the grooves. The projections can be opened by applying pressure to the elastic base using fingers or a surgical tool. The elastic base returns to its original shape after a deformation. The system can also include a device for applying deformation force to the elastic base. An application of deformation force to the elastic base releases pressure on the nerve. An application of deformation force also allows the elastic base to be repositioned.

In accordance with another aspect of the present invention, the system further includes an impedance monitor. The impedance monitor measures impedance in real time. The impedance monitor couples the electrode to a neural stimulator device. The impedance monitor can include a light emitting diode (LED). The LED is configured to indicate whether the electrode has a good connection to the nerve. The impedance monitor further includes a switch. The switch can be engaged to convert the electrode from stimulation and recording mode to impedance monitoring mode. The impedance monitor is configured to deliver short voltage pulses through the electrode. The impedance monitor further includes an op-amp. The op-amp measures output from a voltage divider. A low impedance measurement indicates the electrode is shorted through saline in the body and a high impedance measurement indicates that the electrode is not connected.

### BRIEF DESCRIPTION OF THE DRAWING

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates schematic diagrams of the difference in signals recorded using the previous, common method (the top panel) and the new method (the bottom panel) of the present invention for recordings from the surgically exposed nerve.

FIG. 2 illustrates a perspective view of a sandwich electrode for nerve stimulation, according to an embodiment of the present invention.

FIG. 3 illustrates a perspective view of a sandwich electrode for nerve recording, according to an embodiment of the present invention.

FIG. 4 illustrates a perspective view of another configuration of sandwich electrode for recording, according to an embodiment of a present invention.

FIG. 5 illustrates a perspective view of a sandwich recording electrode with two holding prongs, according to an embodiment of the present invention.

FIG. 6 illustrates a perspective view of a sandwich electrode with a housing tower, according to an embodiment of the present invention.

FIG. 7 illustrates a perspective view of a GMG recording electrode, according to an embodiment of the present invention.

FIGS. 8A-8C illustrate perspective and schematic views of GMG stimulating electrode design, according to an embodiment of the present invention.

FIG. 9 illustrates a schematic diagram of a pen electrode for nerve stimulation, according to an embodiment of the present invention.

FIG. 10 illustrates perspective views of a GMG stimulating electrode from different angles.

FIG. 11 illustrates schematic views of electrode arrangement for NAP recording with sandwich electrodes from surgically exposed peripheral nerve.

FIG. 12 illustrates graphical and image views of an example of ‘non-lifting’ nerve recordings with sandwich electrodes in anesthetized animal.

FIG. 13 illustrates schematic views of electrode arrangement for NAP recording with a combination of a sandwich electrode for recording and a pen electrode for stimulation.

FIGS. 14A-14C illustrate perspective and schematic views of an example of a combined sandwich and pen electrode for ‘non-lifting’ nerve recording in anesthetized animal.

FIG. 15 illustrates a graphical view of an example of NAPs recorded with combined sandwich and pen electrodes.

FIG. 16 illustrates a graphical view of an example of NAPs evoked with a pen electrode in tripolar vs. bipolar mode.

FIG. 17 illustrates a graphical and image view of an example of ‘non-lifting’ nerve recording with GMG electrodes in vitro.

FIG. 18 illustrates a graphical and image view of an example of ‘non-lifting’ nerve recording with GMG electrodes in anesthetized animal.

FIG. 19 illustrates graphical, image and schematic views of a signal specimen and the recording setup with ‘bridge grounding’, according to an embodiment of the present invention.

FIGS. 20A and 20B illustrate graphical views of the test results, according to an embodiment of the present invention.

FIG. 21 illustrates a graphical view of latency shortening as a product of conduction velocity upon reversal of stimulus polarity, according to an embodiment of the present invention.

FIGS. 22A and 22B illustrate graphical views of electrical stimulus artifacts, according to an embodiment of the present invention.

FIGS. 23A-23D illustrate schematic diagrams of how bridge grounding or cut diminishes the artifact, according to an embodiment of the present invention.

FIGS. 24A-24C illustrate graphical and image views of an example of ‘non-lifting’ nerve recording, according to an embodiment of the present invention.

FIGS. 25A and 25B illustrate graphical and image views of an example of ‘non-lifting’ nerve recording in anesthetized animal, according to an embodiment of present invention.

FIGS. 26A-26C illustrate graphical and image views of recording of NAPs with ‘bridge grounding’ in a clinical case, according to an embodiment of the present invention.

FIG. 27 illustrates a schematic view of an RTIM with a connected neural stimulator device and a GMG electrode, according to an embodiment of the present invention.

FIG. 28 illustrates a block diagram of the RTIM, according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims. It should be noted that any dimensions are included simply by way of example and are not meant to be considered limiting. Any suitable dimensions known to or conceivable to one of skill in the art could also be used.

The present invention is directed to a GMG-electrode for recording nerve action potential (NAP) and methods for using such an electrode. The GMG-electrode is so-named for the inventors. Electrophysiological methods are used during repair surgery of peripheral nerve trauma (PNT). PNT is a major medical problem with an annual incidence similar to that of epilepsy. Surgical intervention is provided based on the severity of nerve injury which is determined preoperatively and intraoperatively mainly by electrophysiological assessments. Among those, intraoperative nerve action potential (NAP) or compound action potential (CNAP) recording is preferred for direct assessment of functional continuity of the nerve.

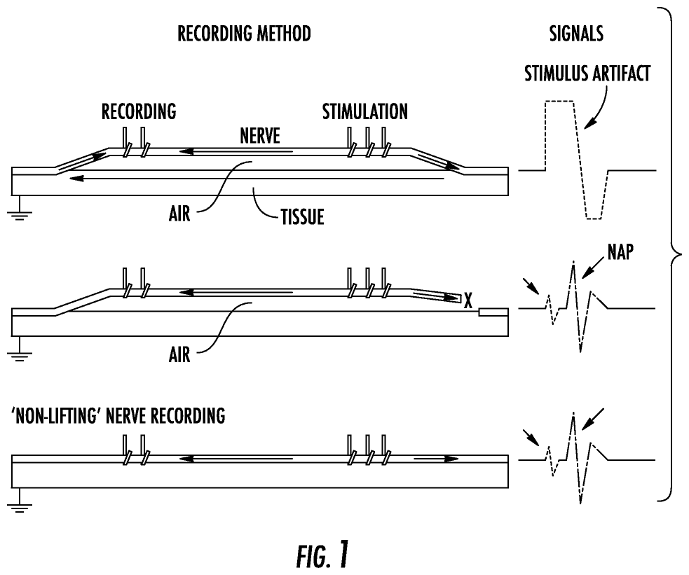

Unsatisfactory, poor intraoperative NAPs associated with large stimulus artifact are recorded with the commonly employed methods using standard hook electrodes. This large stimulus artifact is the consequence of a ‘loop effect’ which is caused by lifting the surgically exposed nerve from the tissue underneath. Based on the loop hypothesis, ‘non-lifting’ nerve recording has been introduced as a new method and been confirmed to result in NAP recordings of satisfactory and significantly improved quality. FIG. 1 illustrates schematic diagrams of the difference in signals recorded using the previous, common method (the top panel) and the new method (the bottom panel) of the present invention for recordings from the surgically exposed nerve. The middle panel shows a piece of evidence for the loop hypothesis on which the new recording method is based. In FIG. 1, stimulus artifacts are in red and neural signals (NAPs) are in blue as indicated by arrows in corresponding colors.

‘Non-lifting’ nerve recordings are essential to obtain interpretable NAP recordings. The currently available nerve electrodes, however, do not easily allow clinicians to record proper NAPs. The newly designed electrodes described in the following material solve the problem with stimulus artifact seen in the clinic.

The innovations for ‘non-lifting’ nerve recording, described below, include 1) new electrodes with self-insulated and self-holding features as well as how to easily apply and remove them from the nerve, and 2) procedures for electrode placement and signal verification (such as the stimulus polarity switch test and the intensity-function test), and instrument parameters.

An electrode according to an embodiment of the present invention includes three metal prongs and one elastic base to which the prongs are attached. It is named a ‘sandwich’ electrode because a nerve is sandwiched between the middle prong and the two outer prongs for stimulation or recording. The elastic or plastic base is orientated in parallel with the nerve to provide the nerve with certain tension when it is sandwiched by the prongs. With appropriate elasticity of the base and appropriate size relationship between the nerve and the prongs, the electrode can be held by the nerve itself without any additional support or hand holding which may excessively stretch the nerve. Another feature of sandwich electrodes is that the metal prongs are insulated except for a small area of contact with the nerve for stimulation or recording. Thus, the nerve does not need to be lifted and can remain in situ to keep good contact with underneath tissue during stimulation and recording to achieve a so called ‘non-lifting’ nerve recording.

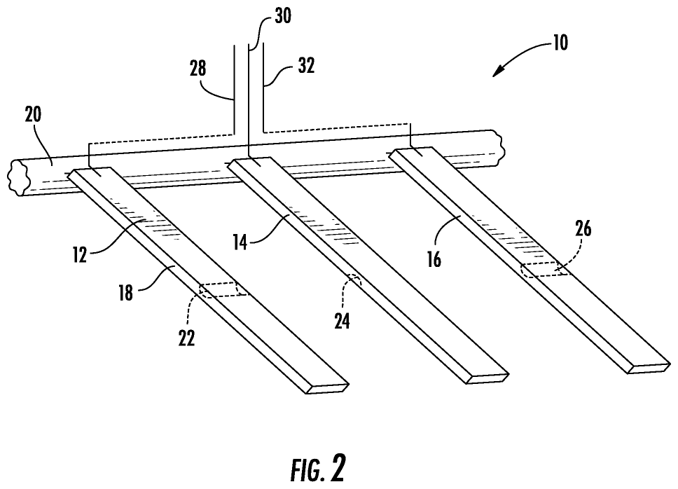

FIG. 2 illustrates a perspective view of a sandwich electrode for nerve stimulation, according to an embodiment of the present invention. In this example, the electrode 10 includes three pins serve as prongs 12, 14, and 16. The prongs 12, 14, and 16 are each insulated with a thin plastic tube 18 and mounted in a plastic base 20. The diameter of the plastic base 20 is approximately 4 mm. The plastic insulation tube of each prong is cut open to create a contact 22, 24, and 26 for the nerve. Contacts 22 and 26 face up and contact 24 faces down. Insulated wires 28, 30, and 32 are connected to prongs 22, 24, and 26 respectively. Wires 28 and 32 are connected to each other, becoming one outlet. Thus, stimulation will be delivered between contact 24 and contacts 22 and 26. The inter-prong distance is 6.5 mm in this example. In a preferred embodiment of the present invention dimensions of each of the prongs 12, 14, and 16 are 1×2×12 mm and dimensions of a contact 22, 24, and 26 are 1×2 mm. Preferably, the pins 12, 14, and 16 are formed with brass, but any other suitable material known to or conceivable by one of skill in the art could also be used.

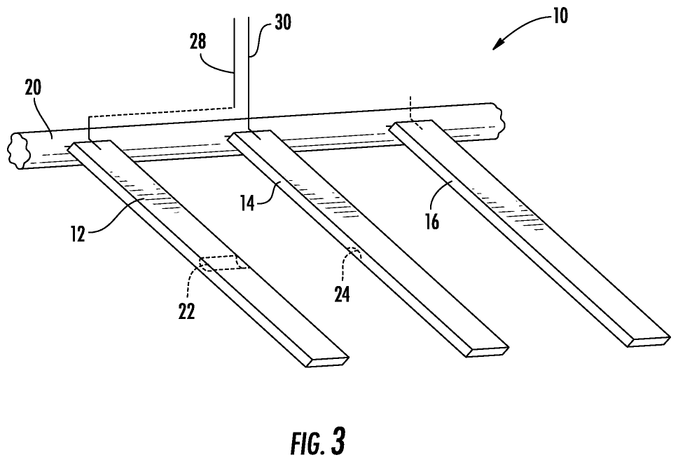

FIG. 3 illustrates a perspective view of a sandwich electrode for nerve recording, according to an embodiment of the present invention. This electrode is identical to the example shown in FIG. 2 except prong 16 is used for holding only and it is not insulated, wires 28 and 30 are not connected to each other, and wires 28 and 30 are braided together. Nerve compound action potentials are recorded between contacts 22 and 24 differentially. Prong 16 can be used as an option for additional grounding. The inter-prong distance is 6.5 mm in this example.

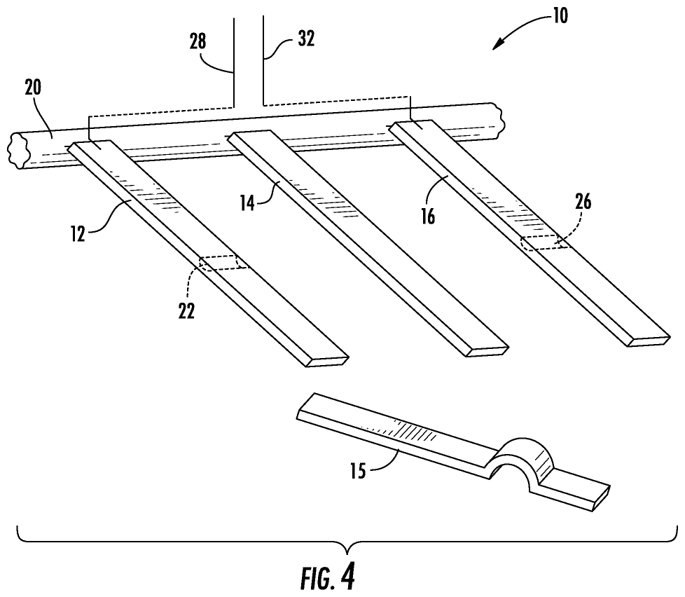

FIG. 4 illustrates a perspective view of another configuration of sandwich electrode for recording, according to an embodiment of a present invention. In this configuration, prong 14 is replaced with a non-metal pin used merely as a holding prong to sandwich the nerve. The holding prong is made of elastic material. Prongs 12 and 16 and their contacts 22 and 26 are identical to those shown in FIG. 3. Wires 28 and 32 are braided together, and a differential recording of compound nerve action potentials is made between contacts 22 and 26. The distance between prongs 12 and 16 is 13 mm in this example. Optionally, a holding prong with a groove (see the insert) can be used instead of the flat holding prong 15 to accommodate a large nerve. The size of the groove can be varied based on the size of nerve.

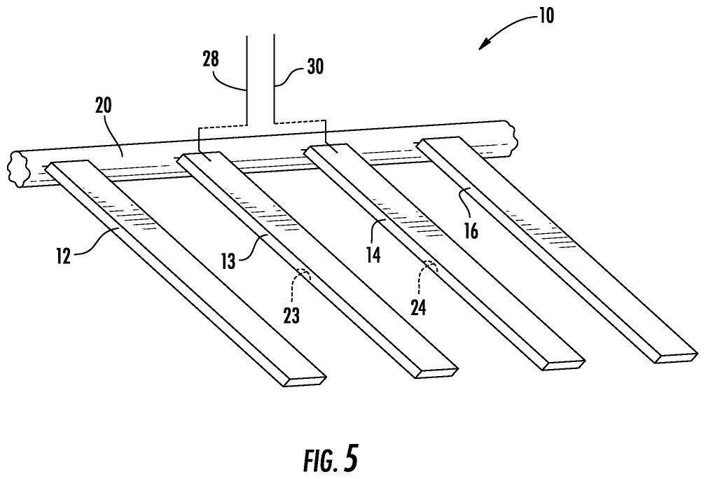

FIG. 5 illustrates a perspective view of a sandwich recording electrode with two holding prongs, according to an embodiment of the present invention. The electrode 10 includes four prongs 12, 13, 14, and 16. The nerve is sandwiched between the two middle electrode prongs 13 and 14 and the two outer holding prongs 12 and 16. Contacts 23 and 24 face downward and the nerve is situated on the underside of prongs 13 and 14. This electrode design allows a short inter-prong distance between prongs 13 and 14 (for instance 5 mm or shorter). In addition, both electrode contacts 23 and 24 are aligned on the same side of the nerve. These two features can enhance the capacity of differential recordings. Furthermore, the two holding prongs are made of uninsulated metal and can be also used to bridge the nerve to the tissue underneath. Wires 29 and 30 are directed to prongs 13 and 14.

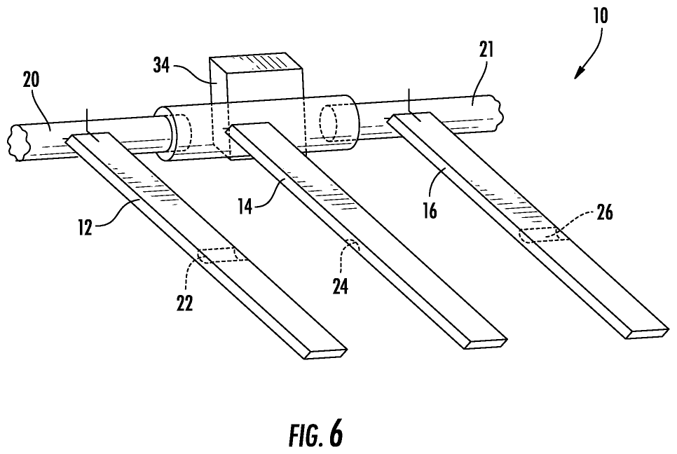

FIG. 6 illustrates a perspective view of a sandwich electrode with a housing tower, according to an embodiment of the present invention. Differential nerve recording is made between contacts 22 and 26. The middle prong 14 is a holding prong. However, a housing block or housing tower 34 is used to house the middle holding prong 14 and the plastic bases 20 and 21 for prongs 12 and 16. The housing tower 34 allows adjustment of inter-prong distance horizontally and/or vertically to accommodate nerves of different size. Horizontal adjustment is made by sliding one or both bases 20 and 21 in or out from the housing tower 34. Vertical adjustment is made by using different slots for the holding prong in the tower. Ideally, a spring can be used in the tower to preset the holding force between the holding prong 14 and prongs 12 and 16.

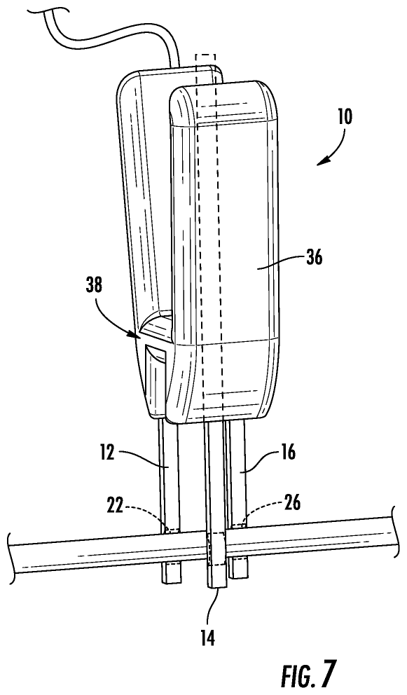

FIG. 7 illustrates a perspective view of a GMG recording electrode, according to an embodiment of the present invention. The electrode 10 includes contacts 22 and 26 facing the nerve and contact 24 is facing away from the nerve. In this embodiment, contact 24 is a reference contact. The recording electrode 10 is composed of a single-molded PDMS silicone clamp (body) 36 with stainless steel planks (prongs) 12, 14, 16 embedded in silicone during the curing process. The steel planks 12, 14, 16 are insulated everywhere except for the electrode contact area. Contacts 22 and 26 are in contact with the nerve whereas contact 24 is not in contact with the nerve but instead faces outward. The height of the bridge 38 is adjusted during fabrication to control the pressure of the electrode (silicone body). A taller bridge 38 will create an electrode with higher pressure between the prongs. Alternatively, contacts 22 and 26 can be arranged in the same way for the sandwich recording electrode as shown in FIG. 3.

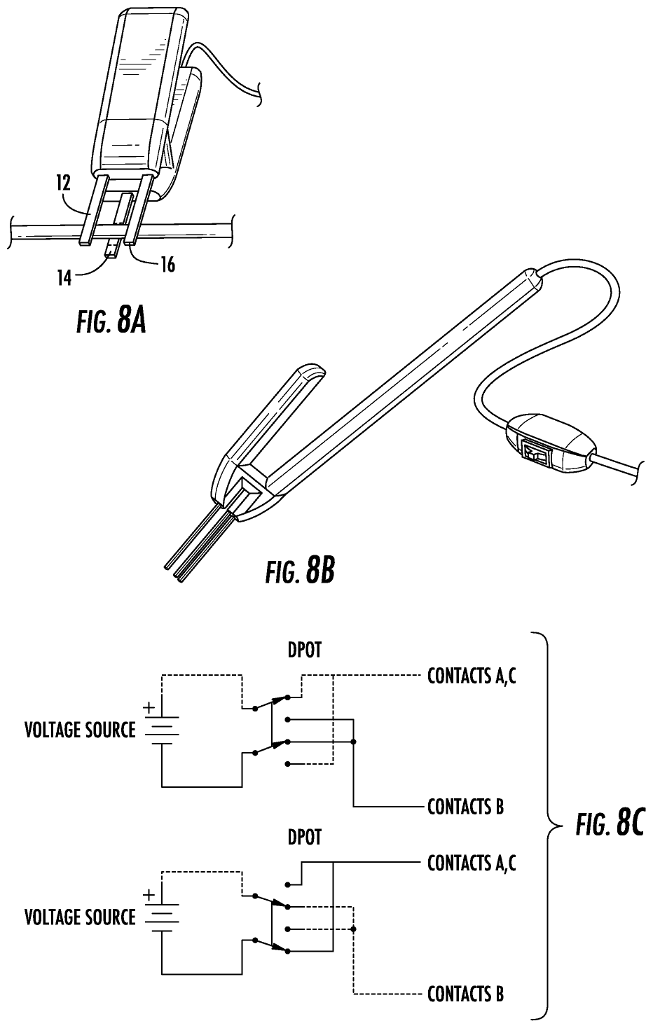

FIGS. 8A-8C illustrate perspective and schematic views of GMG stimulating electrode design, according to an embodiment of the present invention. GMG stimulating electrode allows the surgeon to hold the electrode in one hand, open the clamp and position it on the nerve. FIG. 8A shows the GMG stimulating electrode with no handle. FIG. 8B shows the same electrode with the handle attached. Contacts 12 and 16 are connected together. The DPDT (double pole double throw) switch shown in FIG. 8B and as a schematic in FIG. 8C is connected to the two lines to allow the surgeon to easily switch stimulation polarity.

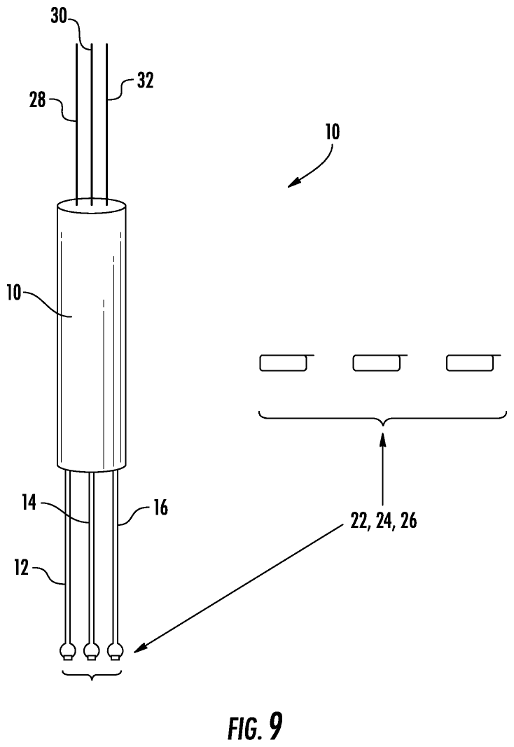

FIG. 9 illustrates a schematic diagram of a pen electrode for nerve stimulation. In this example, three straight metal prongs are housed within a plastic base. The prongs are insulated except for the tips forming contacts 22, 24, and 26. The prongs 12, 14, and 16 are made of stainless steel and the size of rectangle contact for each is 1×2 mm in this example (a bottom enlarged view of prong tips and contacts is shown in the right panel of FIG. 9). The rectangle shape of the contacts 22, 24, and 26 provides a good size of contact and an easy longitudinal alignment with the nerve. The inter-prong distance is 5 mm. Wires 28, 30, and 32 are connected to prongs 12, 14, and 16, respectively, and wires 28 and 32 are connected to form a single outlet. Nerve stimulation is made between contact 24 and contacts 22 and 26. The electrode 10 is held by hand on the plastic base 20 and applied from the top directly to the top surface of the nerve.

Electrodes are disposable and used for acute recordings only. Medical grade stainless steel is a primary option for metal prongs used in the electrodes. Other materials include tungsten, platinum, platinum-iridium alloys, iridium oxide, and titanium nitride. For elastic base, medical grade plastic can be used, and other materials include PDMS silicone.

Dimensions of the electrodes can be adjusted based on the size of nerve to be recorded. These dimensions are included simply by way of example and are not meant to be considered limiting. Any dimensions known to or conceivable by one of skill in the art could also be used. Peripheral nerves from normal children and adults range from 1 to 13 mm in diameter. The horizontal inter-prong distance can be varied from 3 to 7 mm. The vertical distance can be varied from 0 to 9 mm for example. Table 1 lists combinations of different dimensions of the electrodes for different sizes of nerves, for example. A set of these prong dimensions, together with the flexibility provided by the elastic base, provide an electrode with an appropriate fit for a nerve of a given size.

A relatively large contact area is used for all nerve sizes to ensure modest charge-injection densities to address a safety concern with nerve stimulation. Such a concern does not apply to nerve recording and therefore contact sizes can be smaller on recording electrodes.

TABLE 1
Exemplary Dimensions of the Electrodes.

| | Inter-prong distance (mm) | | |
|---|---|---|---|
| Nerve size | Vertical | Horizontal | Contact (mm) |
| Small | 1 | 5 | 1 × 2 |
| Median | 3 | 6 | 1 × 2 |
| Large | 9 | 7 | 2 × 2 |

The stretch and/or pressure on the nerve is less than 20 grams, which is controlled by the elastic base or body of the electrodes. The pressure on the nerve is controlled as for the GMG electrode by adjusting the bridge height during fabrication process.

The electrodes were designed using Solidworks. An electrode clip was cast using suing Slyguard 184 (10:1 curing agent) in a 3-part mold and was cured at 150 C. for 15 minutes Electrodes were insulated with the Same PDMS as the clip design and insulation was removed from appropriate areas. The GMG body of the electrode was molded using GMG design and was tested to ensure it applies less than 10 grams/cm²when applied to the nerve.

The electrodes are used for whole nerve stimulation and compound nerve action potential recording from a surgically exposed peripheral nerve, nerve root, or other neural structure. In peripheral nerve recordings, for example, a nerve segment (about 10 cm or longer) is exposed and isolated from the surrounding tissue. The grounding is placed via a surface or needle electrode positioned near the surgical area. For a good contact with underneath tissue between the two pairs of electrodes it is important that the nerve is kept in situ and not lifted as shown in FIG. 1 (lower panel). Differential recording is made, and input A of the amplifier is connected to the prong of the recording electrode, which is close to the stimulation electrode. In normal stimulus polarity mode, constant current stimulation is delivered from the middle prong (cathode) and the two outer prongs (anode) of the stimulation electrode, whereas the middle prong serves as anode and the two outer prongs as cathode in the reversed stimulus polarity mode. A pair of recordings (one in normal - the other in reversed stimulus polarity mode, but at the same intensity) are acquired sequentially and displayed in the same panel. Stimulus intensities start at 0 mA, allowing to check the background noise level, and then increased to maximally 20 mA (0.05 or 0.1 ms monophasic pulses). Thus, the recording procedures include 1) the stimulus polarity switch test, 2) the intensity-response function test, with the 0 mA control for noise check, and, optionally, 3) steps for nerve inching where the recording site is kept the same while the position of the stimulation electrode is moving stepwise along the nerve.

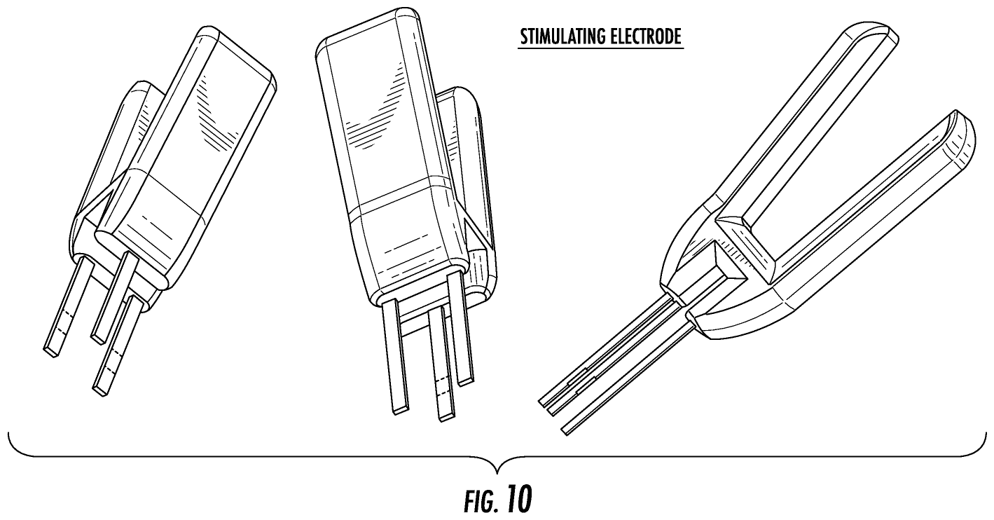

FIG. 10 illustrates perspective views of a GMG stimulating electrode from different angles in detail.

The electrodes are positioned to align all the contacts along the nerve and to fully cover each contact by the nerve. Both stimulation and recording electrodes are free from extra holding by hand or stand. Electrodes with appropriate dimensions are chosen to ensure appropriate stretch/tension on the nerve.

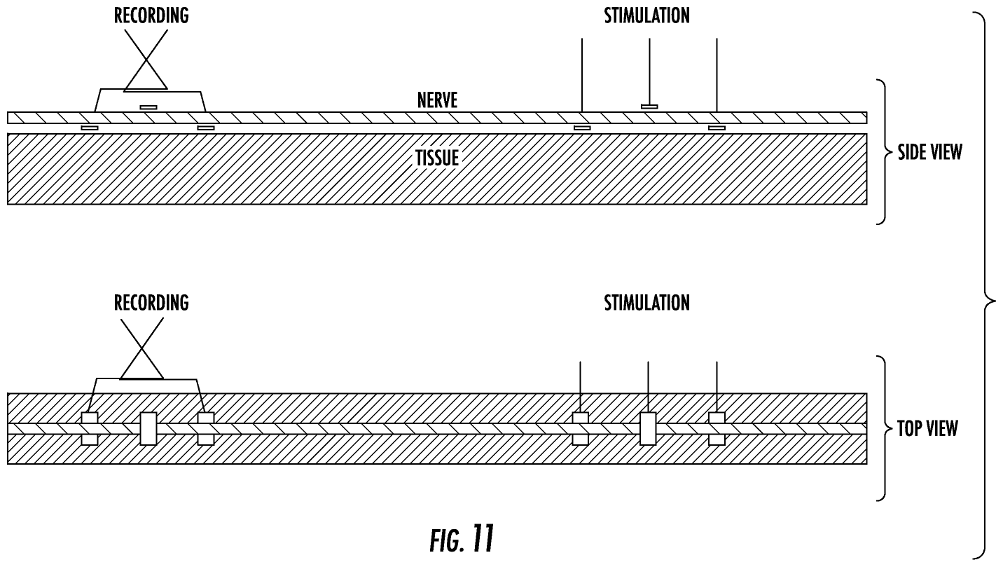

FIG. 11 illustrates schematic views of the electrode arrangement for NAP recording made with sandwich electrodes from surgically exposed peripheral nerve. The upper panel is a side view and the lower panel is a top view of the electrode placements.

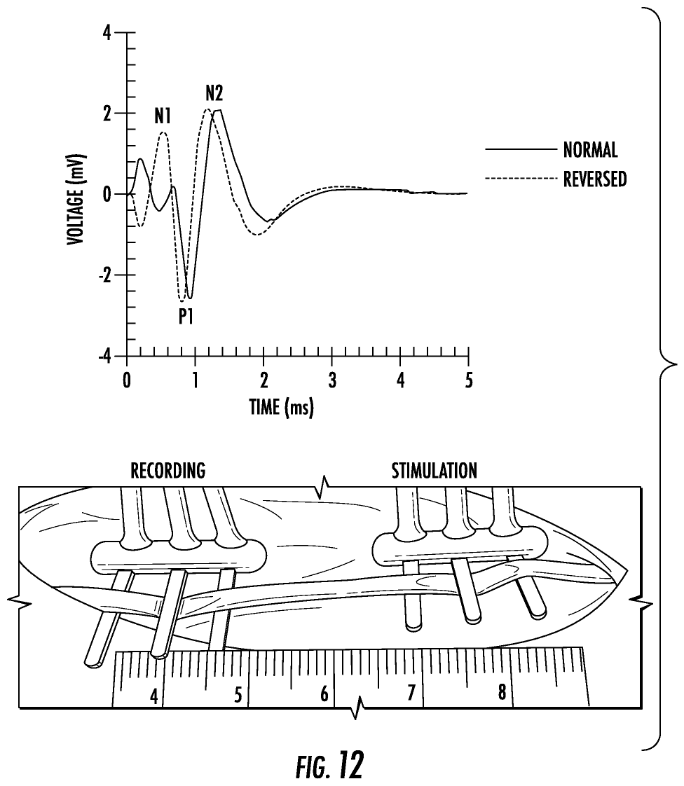

FIG. 12 illustrates graphical and image views of ‘non-lifting’ nerve recordings with sandwich electrodes in anesthetized animal. Two sandwich electrodes were used: one for stimulation (see FIG. 2 for the detail) and the other for recording (see FIG. 3 for the detail). The nerve was positioned between the middle prong and the outer 2 prongs of each electrode in a ‘sandwich’ manner so that the electrodes were held in place by the nerve alone in a hands-free manner without need for additional holding devices. The nerve was not lifted by the electrodes and the nerve segment between the electrodes stayed in good contact with the tissue underneath, thereby achieving a ‘non-lifting’ nerve recording. For stimulation with normal stimulus polarity, the middle prong (i.e., contact B) was a cathode and the two outer prongs (i.e., contacts A and C) were anodes. For stimulation with reversed polarity, the middle prong was an anode and the outer two prongs were a cathode. For recording, the middle prong was connected to input A (i.e., contact A) and the left prong was connected to input B (i.e., contact B) of a differential amplifier. The right prong of the recording electrode was uninsulated and used for holding and keeping the nerve in electrical contact with the tissue underneath. NAPs recorded from this ‘non-lifting’ nerve recording method are shown in the upper panel, FIG. 12. NAP signals are marked with N1, P1 and N2. Stimulus artifacts are small (the voltage deflections prior to NAP signals). Upon reversal of the stimulus polarity (normal: blue trace; reversed: red trace), the stimulus artifact was reversed but not the true NAP signals (P1 and N2), confirming a reliable recording of neural signals. The recording distance was only 31 mm. A constant current stimulus (0.05 ms, 2 mA) was used. The bandwidth of the filter was 120 Hz to 3 kHz.

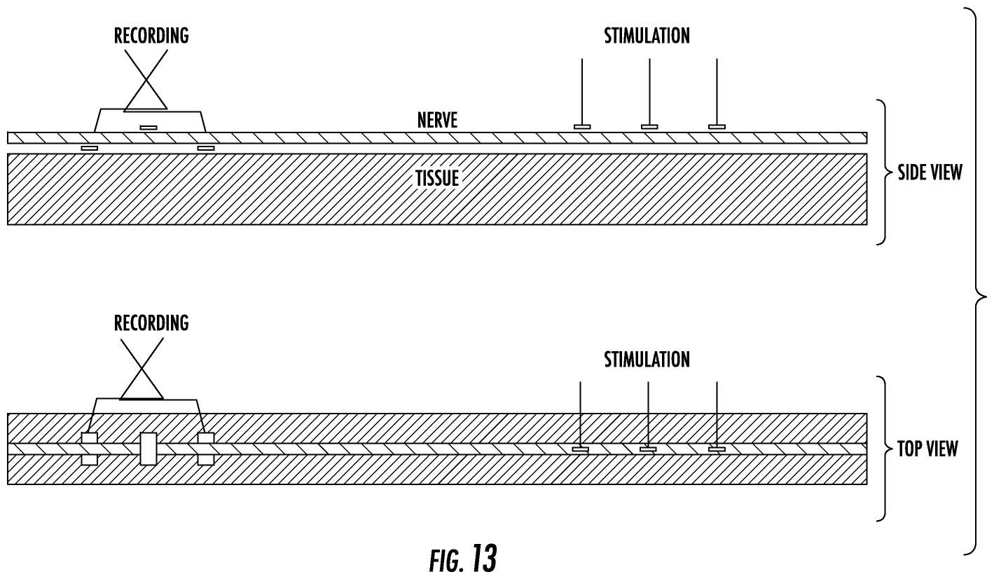

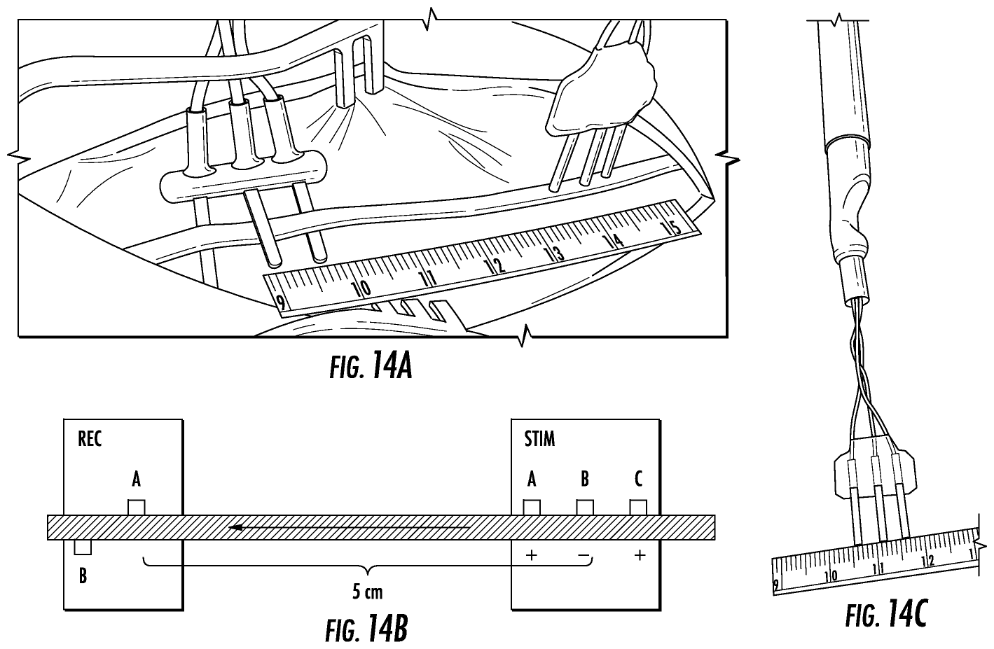

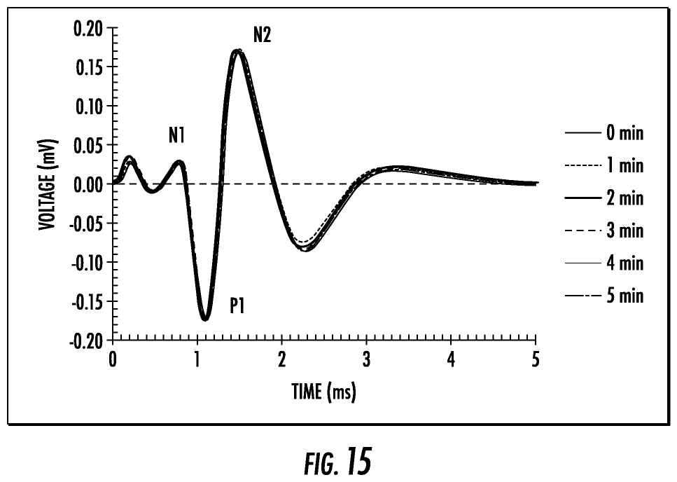

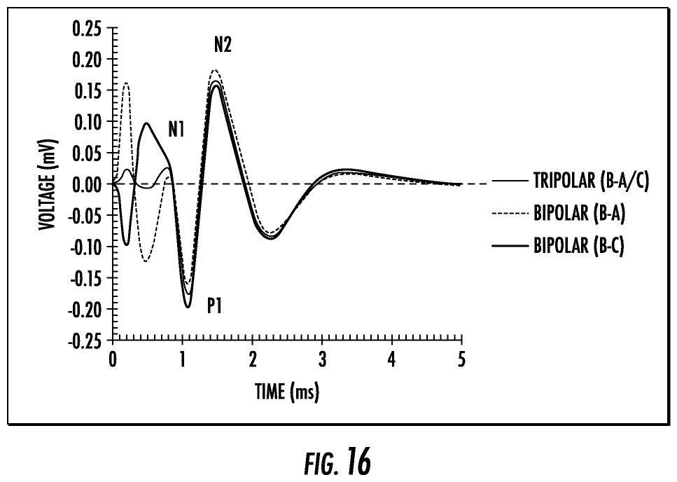

FIGS. 13-16 show an example of NAP recordings with combined sandwich and pen electrodes from surgically exposed peripheral nerve. FIG. 13 illustrates schematic views of the electrode arrangement: the upper panel is a side view and the lower panel is a top view. FIGS. 14A-14C illustrate an example of such recordings in anesthetized animal. The placement of the electrodes is shown in FIG. 14A. The recording electrode was placed in the same way as described in FIG. 12. The pen stimulation electrode was held by hand and applied to the top surface of the nerve under an operating microscope. The nerve was dissected and isolated from the surrounding tissue but was not lifted. The nerve segment between the two electrodes stayed in good contact with the tissue underneath, thereby achieving a ‘non-lifting’ nerve recording. Connections of the electrodes to the amplifier and the stimulator are shown in a schematic drawing in FIG. 14B. A photograph of the pen electrode is also shown in FIG. 14C. In the pen electrode, three prongs were insulted except for their tips, and inter-prong distance was 5 mm in this example. NAPs recorded from stimulation with normal stimulus polarity are shown in FIGS. 15-16. NAP signals are marked with N1, P1 and N2. In FIG. 15, six recordings were taken over a 5 min period of continuous recording to show the good stability of the recordings from this method with tripolar stimulation. In FIG. 16, three recordings are shown to illustrate the advantage of the tripolar stimulation over the bipolar stimulation for minimizing the size of stimulus artifact, which is in line with the previous report. Again, NAP signals are marked with N1, P1 and N2, and voltage deflections prior to NAP signals are stimulus artifacts. In all of these recordings, the recording distance was 50 mm, the same constant current stimulus was used (0.05 ms, 2 mA, normal polarity), and the filter bandwidth was 120 Hz to 3 kHz.

The recording method using a combination of a sandwich electrode for recording and a pen electrode for stimulation keeps the feature of ‘non-lifting’ nerve recording and improves the flexibility for moving the stimulation site along the nerve. Moving stimulation site along the nerve is performed in nerve inching to collect NAP data around the nerve injury site to localize the site of conduction failure.

The recording and stimulation configurations when using GMG electrodes are the same as those for sandwich electrodes. However, GMG electrodes are designed to be easily applied to and removed from the nerve and for reducing application variability when they are applied to peripheral nerves simply by pressing and releasing the clip. Thus, GMG electrodes can be easily released and moved to a different location along the nerve. In the same way, GMG electrodes can also be used to standardize the peripheral nerve recording procedures for improved consistency in single or repetitive electrode applications. ‘Non-lifting’ nerve recording of clean NAPs using GMG electrodes has been demonstrated in in vitro and in vivo experiments conducted on isolated and exposed animal peripheral nerves.

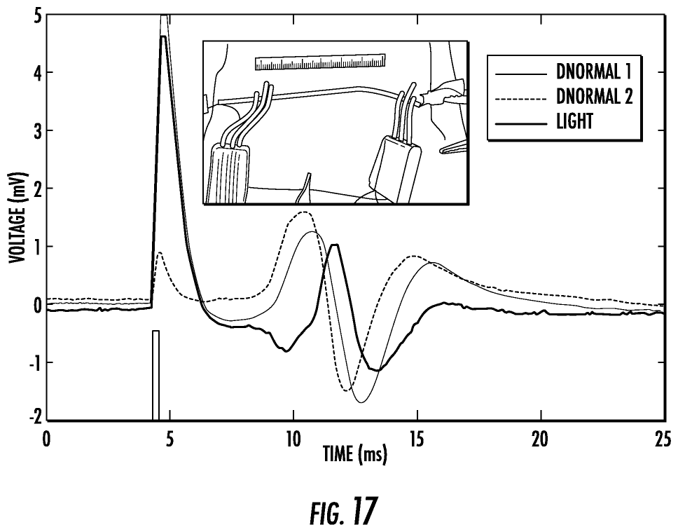

FIG. 17 illustrates a graphical and image view of an example of ‘non-lifting’ nerve recording with GMG electrodes in vitro. FIG. 17 shows NAPs recorded in vitro with GMG electrodes from the isolated animal peripheral nerve. Recordings were made in a model system. In the system, gauze, soaked with synthetic interstitial fluid, was placed on a platform mimicking the surrounding tissue and body. The GMG recording electrode used here had two active contacts: A and B (two outer prongs); and one reference contact (middle prong): REF (see FIG. 7 for detail). Differential recordings (two channels) were made from contact A and contact B, respectively, with a common reference. Three voltage traces are shown: blue (contact A), yellow (contact B), and red (contact A-contact B). To achieve similar amplitudes of signals on both channels, different amplifications were used: 3500 and 10000. The bandpass filter: DC to 1000 Hz (12 dB). A constant current stimulus (0.05 ms, 2 mA) was delivered with normal polarity (stimulus is indicated by a blue vertical line in FIG. 17). The GMG stimulating electrode had three contacts (see FIG. 8A for detail): contact B (anode) and contacts A/C (cathode). The recording arrangement is shown in the insert of FIG. 17: recording (left) and stimulating (right) electrodes.

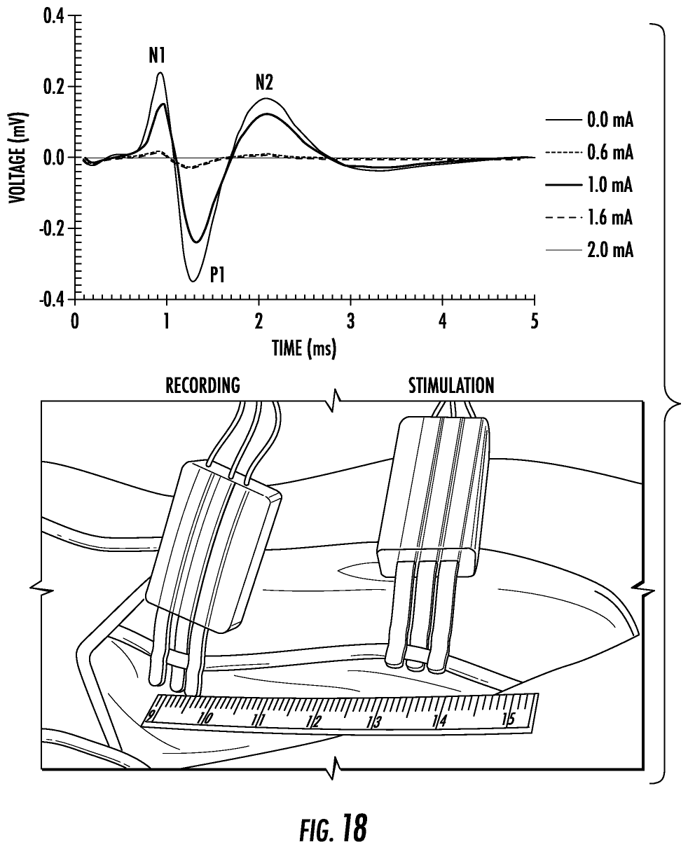

FIG. 18 illustrates a graphical and image view of an example of ‘non-lifting’ nerve recording with GMG electrodes in an anesthetized animal. Two GMG electrodes were used: one for stimulation and the other for recording. The placement of the electrodes is shown in the lower panel. The nerve segment between the electrodes was not lifted but stayed in contact with the surgical bed, thereby achieving a ‘non-lifting’ nerve recording. NAPs are shown in the upper panel. NAP signals are marked with N1, P1 and N2. Differential recording (single channel) was made between contacts A and B of the GMG recording electrode (see FIG. 7 for detail). Constant current stimuli (0.05 ms) of intensities varied from 0 to 2 mA were used. NAP signals showed saturation in amplitude at an intensity of 1.6 mA. The recording distance was 45 mm.

A differential amplifier and an isolated constant current stimulator (biomedical safety) are two major components of equipment. The bandwidth is 120 Hz to 2.5 kHz (10 Hz to 3 kHz, optional). An analog or digital stimulus polarity switch is provided. Impedance measurement and/or control of electrodes can be implemented. The electrodes are connected to the headbox of a standard IONM (intraoperative neurophysiological monitoring) machine.

In exemplary implementations of the present invention, which are not meant to be considered limiting, but are included as further illustration of the invention, five (3 females and 2 males) adult pigtail monkeys (Macaca nemestrina) weighing 7 to 20 kg were used for recordings under anesthesia. From two additional male monkeys, weighing 4 kg each, peripheral nerves were acquired postmortem and used for recordings in a model system.

Following initial sedation with intramuscular ketamine (12 mg/kg, Phoenix Pharmaceutical, Inc., St., Joseph, Miss.), anesthesia was induced by an intravenous bolus of pentobarbital (6 mg/kg, Ovation Pharmaceuticals, Inc., Deerfield, Ill.), and animals were intubated. Anesthesia was maintained by pentobarbital (continuous infusion at 4-6 mg/kg/h) or isoflurane (0.5-2%). Neuromuscular blockade (NMB) was induced and maintained with pancuronium bromide (0.1 mg/kg every 2 h, SICOR Pharmaceuticals, Irvine, Calif.). Animals were ventilated to maintain a pCO2 of 35-40 mmHg. Heart rate was monitored with an ECG. Core temperature was maintained near 38° C. using feedback-controlled warm-water heating pads. An intravenous drip of 5% dextrose was continuously administered.

Using aseptic techniques, an incision was made on the upper arm. A segment of 10-15 cm length of either median or ulnar nerve was exposed and, under a microscope, carefully dissected from the surrounding tissue while keeping the epineurium intact. During and after dissection, the nerve was irrigated frequently with normal saline solution.

Compound action potentials were recorded from the whole nerve. The animal was grounded through a needle electrode positioned subcutaneously near the incision. Signals were filtered (low frequency 120 Hz; high frequency 3 kHz; Krohn-Hite 3700 Filter, Krohn-Hite Corp., Avon, Mass.), amplified differentially (EG&E Princeton Applied Research 5113 Amplifier, Ametek, Berwyn, Pa.), and digitized at a sampling rate of 25 kHz (Digital Acquisition Processor board, Microstar Laboratories Inc.). Gain varied from 1000 to 25,000. Signals were recorded on a PC using DAPSYS software (v.8; Brian Turnquist, see www.dapsys.net). The nerve was stimulated with constant current, monophasic square pulses (0.05 or 0.1 ms, up to 20 mA, Digitimer DS7A Stimulator, Digitimer Ltd., Welwyn Garden City, England) delivered at 0.25 Hz (controlled by DAPSYS). No signal averaging was carried out for recordings.

Standard IONM hook electrodes (Cadwell, Kennewick, Wash.) and in-house made ‘self-holding’ electrodes were used to perform bipolar recording and tripolar stimulation. The stimulation electrode was either placed underneath the nerve or used to ‘sandwich’ the nerve. For sandwiching, the middle prong of the IONM electrode was bent upwards. The inter-prong distance of IONM electrodes was 6 and 5 mm for recording and stimulation, respectively. The in-house made electrodes for stimulation and recording had the same inter-prong distance (6.5 mm). The two outer prongs were connected to a common outlet and served as an anode while the middle prong served as a cathode for tripolar stimulation with normal stimulus polarity. For the stimulation with reversed polarity, the two outer prongs served as a cathode and the middle prong anode. Standard recording and stimulating electrodes were held by magnetic stands during most recordings.

In a simple model system, saline-soaked gauze (4″×4″) was placed on a platform mimicking the surrounding tissue and body. A saline-soaked gauze strip (three strand braid, 10-12 cm long and 3 mm in diameter) or segment of actual nerve was held by recording and stimulation electrodes such that it was suspended in the air between electrodes, but that the ends touched the ‘tissue’ underneath. This arrangement therefore mimics the recording situation in the operating room. Electrodes, equipment, parameters and configurations were identical to those used for the animal experiments. The system was grounded through a clipper electrode attached to the ‘body’. For nerve recordings in the model system, saline was replaced with synthetic interstitial fluid composed as described previously. Recordings were made at room temperature (about 24° C.).

The findings were applied to an adult patient in whom routine IONM procedures were used to evaluate a left brachial plexus injury during surgical nerve repair. The patient was under general anesthesia with no NMB agents given after induction. Intraoperative NAPs were recorded with an IONM machine (Neuromaster MEE-2000 Intraoperative Monitoring System; Nihon Kohden, Tokyo, Japan). The settings were 100 Hz and 3 kHz for low- and high frequency filter, respectively. The sampling rate was 10 kHz. The same type of Cadwell IONM electrodes were used for nerve stimulation and recording but were held by hand. Constant current stimulation was triggered manually. Stimulation duration was 0.1 ms (monophasic square pulse) and intensity was 0, 2, 5 or 10 mA. The patient was grounded via a sterile subdermal needle electrode (Rhythmlink, Columbia, S.C.) positioned in the shoulder.

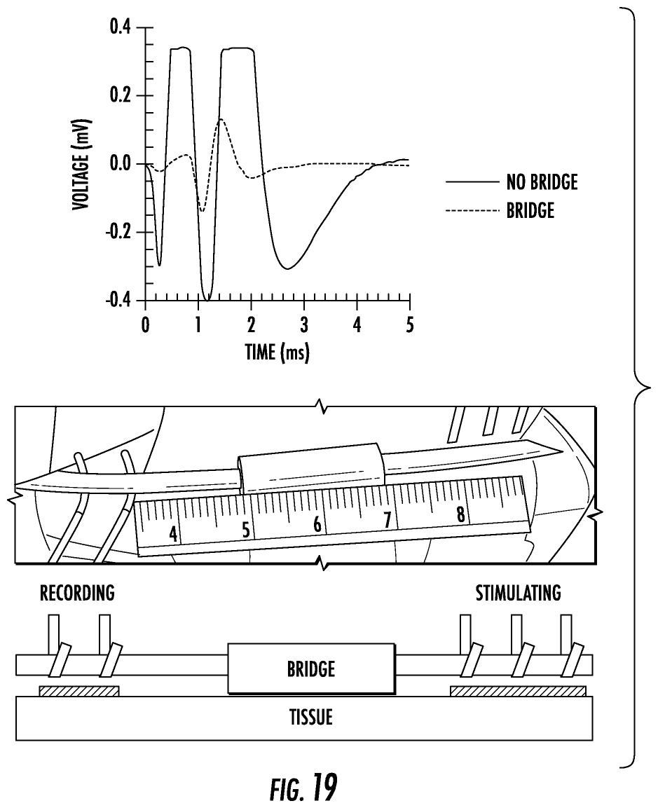

Recordings were made using standard IONM hook electrodes from either median or ulnar nerve in the upper arm of the anaesthetized monkey with full NMB. Following the recommended method, stimulation and recording electrodes were used to lift the nerve from the tissue underneath. A large electrical artifact contaminated or completely obscured the neural signal in all recordings performed in this manner. The stimulus artifact was suppressed and a NAP became recognizable after placing a saline-soaked gauze underneath the nerve between the stimulating and recording electrodes to bridge the nerve and the surrounding tissue (i.e., ‘bridge grounding’). Signal specimen and the recording setup are shown in FIG. 19. FIG. 19 illustrates graphical, image and schematic views of a signal specimen and the recording setup, according to an embodiment of the present invention. Results from all five experiments are summarized in Table 2 where the recordings were made with or without ‘bridge grounding’. With no exception, ‘bridge grounding’ was needed in these recordings to suppress the large stimulus artifacts and unmask NAPs. NAPs recorded with ‘bridge grounding’ in the five experiments are summarized in Table 3. Recording distances varied in the recordings, ranging from 16 to 51 mm (an average of 42 mm, n=75). In FIG. 19, ‘Bridge grounding’ unmasks NAP signals from large stimulus artifacts in recordings from the peripheral nerve of the anesthetized monkey. The figure includes examples of recordings with or without ‘bridge grounding’. The recordings were made from the median nerve in the upper arm. The recording distance was 49 mm, and a constant current stimulus (0.05 ms, 5 mA) was applied. Without “bridge grounding”, a large stimulus artifact was recorded (blue trace). With the “bridge grounding”, the artifact was suppressed, and the NAP signal emerged (red trace). The figure also includes a photograph and schematic drawing showing the recording setup with ‘bridge grounding’. IONM electrodes, including a double hook recording electrode and a triple hook stimulating electrode, were applied to the nerve. Saline-soaked gauze was placed around the nerve between the stimulating and recording electrodes to bridge the nerve and the surrounding tissues, serving as ‘bridge grounding’. A piece of insulation material was placed underneath each of the electrodes to isolate them from the surrounding tissue.

TABLE 2
Unmasking of NAP signals from large stimulus artifacts by ‘bridge grounding’
was observed in all recordings from the peripheral nerves of the anesthetized monkeys.

| | | Body | | Recording | | |
|---|---|---|---|---|---|---|
| | | weight | | distance | Without ‘bridge | With ‘bridge |
| Monkey ID | Sex | (kg) | Nerve | (mm) | grounding’ | grounding’ |
| 43R | Female | 7 | Median | 23 to 30 | Large stimulus | Distinct NAP |
| | | | | | artifact | |
| 77A | Male | 8 | Ulnar | 38 to 51 | Large stimulus | Distinct NAP |
| | | | | | artifact | |
| Lf2 | Male | 20 | Median | 48 to 51 | Large stimulus | Distinct NAP |
| | | | | | artifact | |
| AR10 | Female | 12.5 | Ulnar | 16 to 40 | Large stimulus | Distinct NAP |
| | | | | | artifact | |
| 26X | Female | 7.5 | Ulnar | 21 to 41 | Large stimulus | Distinct NAP |
| | | | | | artifact | |

TABLE 3
Measurements of NAPs (n = 75) recorded with ‘bridge grounding’
from the peripheral nerves of the five anesthetized monkeys.

| | Peak latency | Peak amplitude | CV |
|---|---|---|---|
| NAP | (ms) | (mV) | (m/s) |
| N1 | 0.72 ± 0.02 | 0.30 ± 0.04 | 57.5 ± 1.0 |
| | (0.69-0.84) | (0.02-0.47) | (51.7-63.0) |
| P1 | 1.06 ± 0.03 | −0.63 ± −0.05 | 38.5 ± 0.8 |
| | (1.02-1.17) | (−1.08-−0.14) | (31.9-45.9) |
| N2 | 1.56 ± 0.04 | 0.45 ± 0.04 | 26.8 ± 0.7 |
| | (1.44-1.80) | (0.12-0.61) | (21.4-34.7) |

Mean ± SEM (25%-75% percentile). CV, conduction velocity.

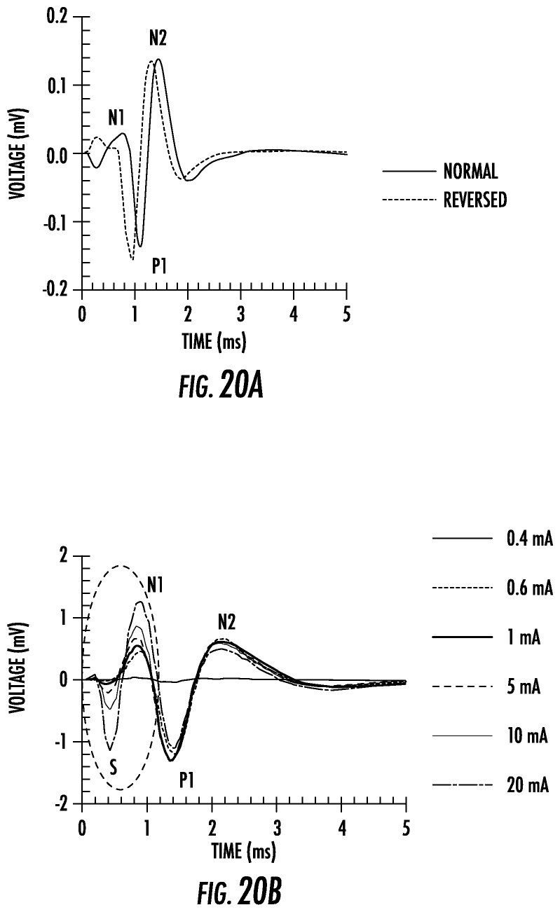

FIGS. 20A and 20B illustrate graphical views of the test results, according to an embodiment of the present invention. NAP signals were further confirmed by: 1) the stimulus polarity switch test, and 2) the intensity-response function test. These allow the investigator to differentiate neural signals from electrical artifacts. The first test is based on the principle that electrical artifacts, but not neural signals, change polarity when stimulus polarity changes. The second test is based on the principle that the amplitude of neural signals, but not the stimulus artifacts, saturates with increasing stimulus intensity. In the recordings with ‘bridge grounding’, NAP signals (three peaks marked as N1, P1 and N2) were clearly identified using these tests (see FIGS. 20A and 20B). However, even with ‘bridge grounding’, stimulus artifact still obscured the early component(s) of the NAP, like N1, especially at high stimulus intensities (FIG. 20B). Upon reversal of stimulus polarity, the latency of NAP became shorter (FIG. 20A).

In FIGS. 20A and 20B, verification of NAP signals in recordings from the peripheral nerve of the anesthetized monkey. FIG. 20A illustrates an effect of switching the stimulus polarity. The sample trace was taken from the same recording with the ‘bridge grounding’ shown in FIG. 20A. Upon reversal of the stimulus polarity (normal: blue trace; reversed: red trace), the stimulus artifact (the voltage deflection prior to N1) was reversed but not the NAP waveform (N1, P1, and N2). Note that the latency of the NAP was shortened by reversing the stimulus polarity. For the normal (reversed) stimulus polarity the middle prong was cathode (anode) and the outer 2 prongs were anode (cathode). FIG. 20B shows an effect of varying the stimulus intensity. The specimen recording was from the ulnar nerve in the upper arm. The size of the stimulus artifact (early deflections circled by a dash line: S and N1) increased with stimulus intensity whereas NAP signals showed saturation (P1 and N2). The recording distance was 51 mm, and the stimulus duration was 0.1 ms.

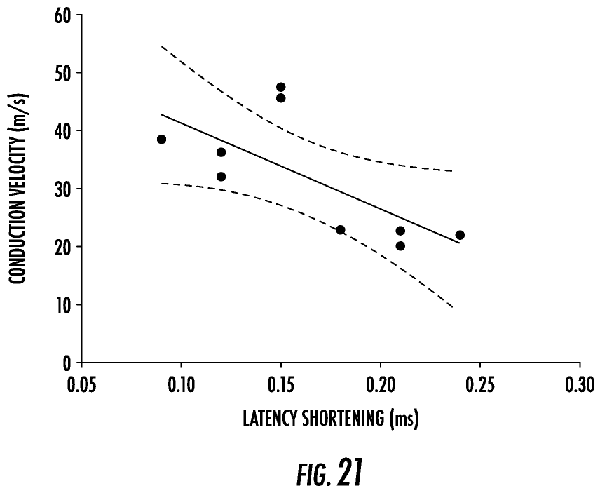

FIG. 21 illustrates a graphical view of latency shortening as a product of conduction velocity upon reversal of stimulus polarity, according to an embodiment of the present invention. The amount of latency shortening induced by reversing stimulus polarity seems to be correlated negatively with the nerve conduction velocity (r=−0.699, P=0.036, FIG. 21). In addition, the threshold for maximal NAP slightly increased upon polarity reversal (Table 4). As shown in Table 4, a stimulus intensity that induced a maximal NAP (represented by peak P1 amplitude) with normal stimulus polarity failed to produce a maximal response with the reversed stimulus polarity. The maximal response was elicited with the reversed polarity when the intensity was increased, however.

FIG. 21 illustrates the correlation between latency shortening of NAPs upon reversal of stimulus polarity and nerve conduction velocity. Data were obtained in recordings from monkey peripheral nerves. Peak latencies of P1 are used in analysis. The solid line indicates linear regression, and the two dash lines indicate the regression band (95% confidence interval). r=−0.699, P=0.036, n=9. The analysis was performed using Statistica 6.1 (StatSoft Inc., Tulsa, Okla.).

TABLE 4
Stimulus intensity for maximal NAP increased slightly upon reversal of stimulus
polarity in recordings from the peripheral nerves of the anesthetized monkeys.

| | Normal stimulus polarity | | Reversed stimulus polarity | | |
|---|---|---|---|---|---|
| | Intensity | Amplitude | Intensity | Amplitude | Fold increase |
| Recording | (mA) | (P1, mV) | (mA) | (P1, mV) | of intensity |
| 1 | 1.5 | −0.13 | 1.5 | −0.02 | 2.0 |
| | | | 3.0 | −0.14 | |
| 2 | 1.5 | −0.45 | 1.5 | −0.18 | 1.3 |
| | | | 2.0 | −0.35 | |
| 3 | 1.0 | −0.28 | 1.0 | −0.12 | 2.0 |
| | | | 2.0 | −0 32 | |
| 4 | 1.0 | −0.29 | 1.0 | −0.02 | 2.0 |
| | | | 2.0 | −0.33 | |

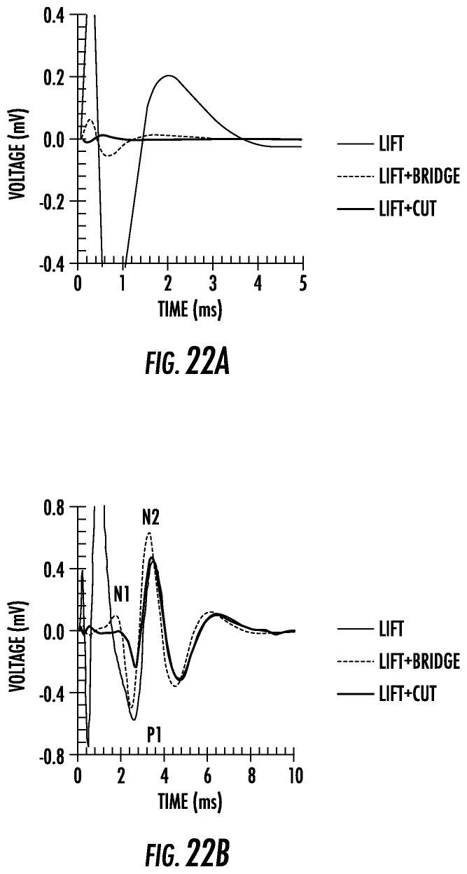

FIGS. 22A and 22B illustrate graphical views of electrical stimulus artifacts in a model system, according to an embodiment of the present invention. Exaggerated stimulus artifacts might be caused by a loop effect due to lifting the nerve segment between the recording and stimulation electrodes. To test this hypothesis, a simple model system in which the loop can be mimicked and, more importantly, opened is used. Such test could not be performed in animal recordings. In this model system, a large stimulus artifact was recorded when the ends of a gauze strip (FIG. 22A) or an actual nerve (FIG. 22B), suspended between stimulation and recording electrodes, touched the ‘tissue’ underneath. The artifact was suppressed when the ‘bridge grounding’ was applied. Strikingly, the artifact was substantially diminished when one end of the gauze strip or actual nerve was lifted from the ‘tissue’ underneath (‘cut’). The hypotheses regarding how ‘bridge grounding’ or ‘cut’ diminishes the artifact are illustrated in FIGS. 22A and 22B: when the nerve is lifted but its ends are in contact with the ‘tissue’, a loop forms within which electrical currents travel to the recording electrode, resulting in exaggerated artifacts.

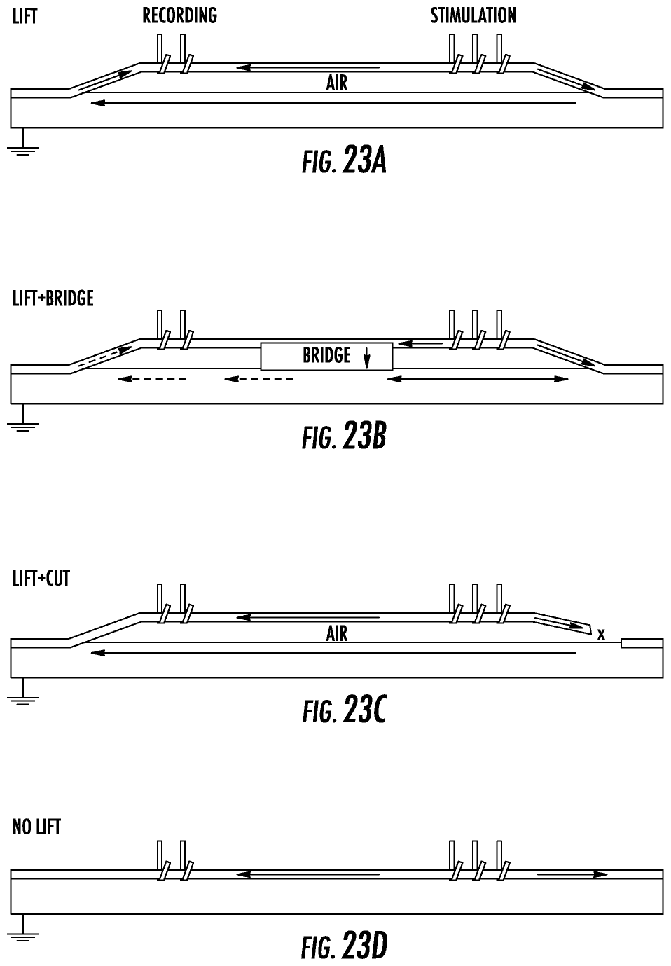

FIGS. 22A and 22B illustrate evidence obtained in a model system supports the loop hypothesis regarding the exaggerated stimulus artifacts encountered in NAP recordings. FIG. 22A illustrates stimulus artifacts recorded in the model system without nerve. The nerve and tissue were mimicked by saline-soaked gauze. A large stimulus artifact was recorded when the ‘nerve’ was lifted up in the air (blue trace). The stimulus artifact was suppressed when the ‘bridge grounding’ was applied (red trace) or even more diminished by ‘cutting’ the connection between the ‘nerve’ and the ‘surrounding tissue/body’ (green trace). The recording distance was 60 mm, and a constant current stimulus (0.05 ms and 2 mA) was applied. FIG. 22B illustrates signals recorded in the model system using a peripheral nerve segment (monkey tibial nerve, about 10 cm). Again, a large stimulus artifact was recorded when the nerve was lifted up in the air but suppressed by ‘bridge grounding’ or following a ‘cut’. In contrast, NAP signals (N1, P1 and N2) underwent little change upon applying the ‘bridge grounding’ or after ‘cut’. The recording distance was 50 mm, and a constant current stimulus (0.05 ms, 1 mA) was applied. The recording setup and experimental conditions are schematically illustrated in FIG. 23A-23D.

FIGS. 23A-23D illustrate schematic diagrams of how bridge grounding or cut diminishes the artifact, according to an embodiment of the present invention. ‘Bridge grounding’ disturbs the loop, and consequently suppresses the artifact. When the loop is ‘cut’ open, the artifact is diminished. FIG. 23D suggests that the best recording result is expected when the nerve is not lifted because no loop forms.

Further, FIGS. 23A-23D illustrate schematic drawings of the recording setup and experimental conditions used in FIGS. 22A and 22B. The nerve lies on the tissue, and arrows show directions of current flow. The grounding is made through the tissue. NAP recordings are made with a bipolar hook electrode and stimulation by a tripolar hook electrode. In FIG. 23A the nerve is lifted (loop is formed). In FIG. 23B, the nerve is lifted but connected to the underneath tissue by a ‘bridge’ (loop is disturbed). In FIG. 23C, the nerve is lifted but ‘cut’ at the end close to the stimulation (loop is broken). In FIG. 23D, the nerve is not lifted (loop is not present).

The model system recordings using a nerve demonstrate that the loop effect influences stimulus artifacts but not neural signals. This might be explained by the fact that these two types of signals travel differently in the loop with neural signals reaching the recording electrode only by one-way conduction along the nerve.

Based on the loop hypothesis, intraoperative NAP recordings were thought to significantly improve if the nerve is not lifted. ‘Non-lifting’ nerve recordings in the model system using modified IONM electrodes were executed with prongs fully insulated except for an area to be in contact with the nerve. This abolished the need to lift the nerve or use the ‘bridge grounding’ technique.

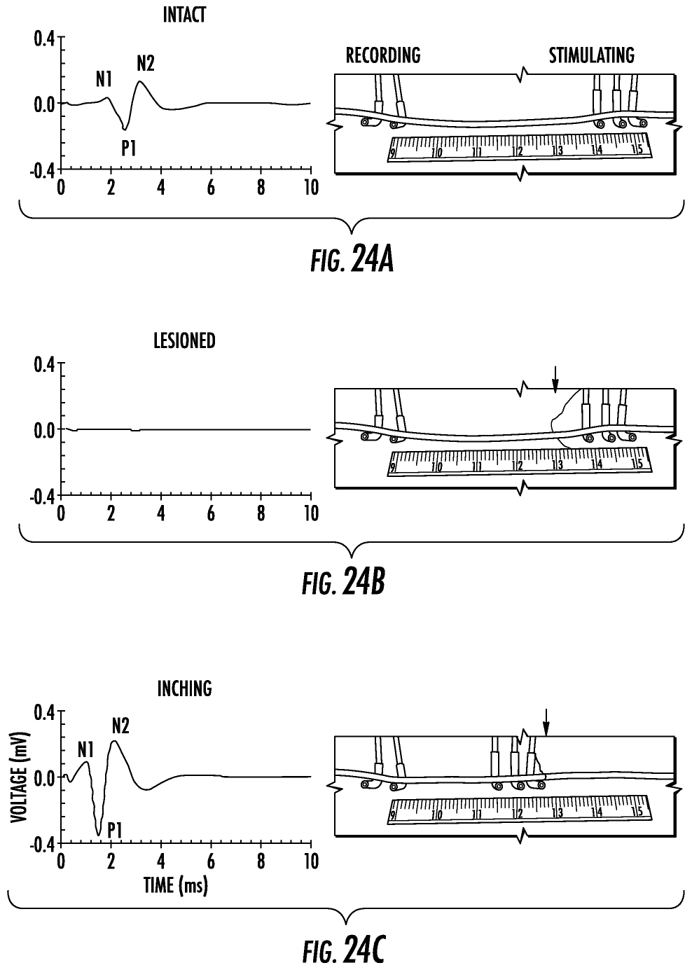

In FIGS. 24A-24C, ‘non-lifting’ nerve recording was made using modified IONM electrodes in the modal system. The recordings were made from the tibial nerve. NAP recordings are shown to the left and electrode arrangements to the right. Clean NAP was recorded when the nerve was not lifted, as illustrated in FIG. 24A. NAP signals were lost after the crush lesion was induced by ligating the nerve with a suture (indicated with a red arrow), as illustrated in FIG. 24B. NAPs returned when the stimulating electrode was moved distally (as illustrated in FIG. 24C, inching). The stimulation (0.05 ms, 2 mA) and other parameters were kept unchanged throughout recordings. Normal stimulus polarity was used.

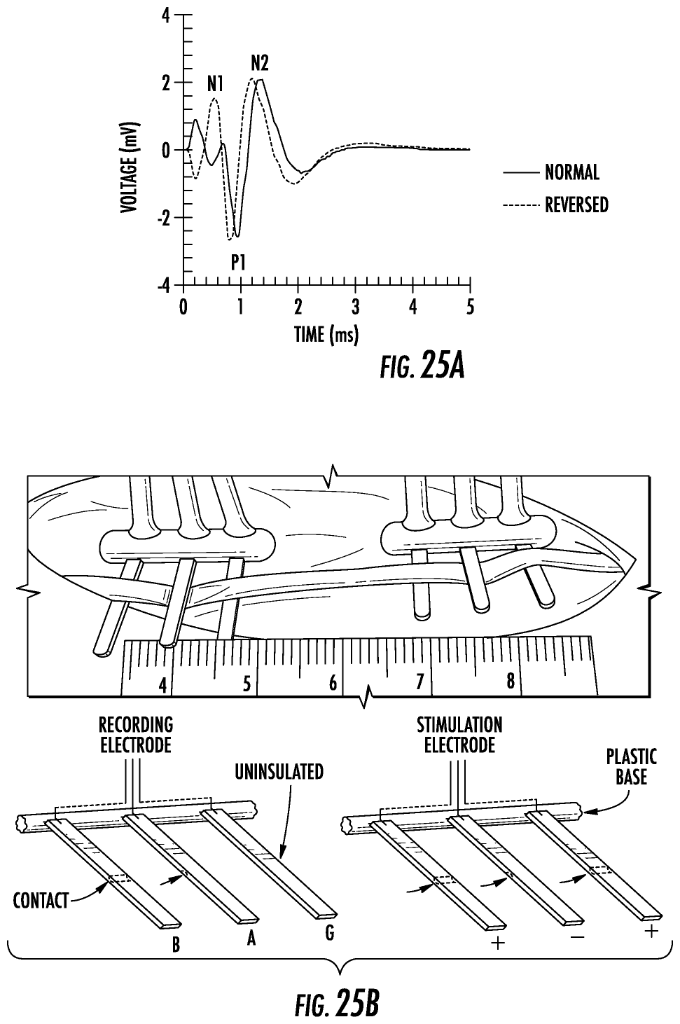

‘Non-lifting’ nerve recordings were performed in an anesthetized monkey with newly designed, in-house made ‘self-holding’ sandwich electrodes. As shown in FIGS. 25A and 25B, there is no need to lift the nerve from the tissue underneath because these electrodes are self-insulated and self-held. In recordings shown in FIG. 25A, NAPs were recorded and separated from stimulus artifacts without ‘bridge grounding’. No additional fixation of the electrodes was necessary, and the nerve stayed in good contact with the tissue underneath as shown in FIG. 25B, thereby achieving a ‘non-lifting’ nerve recording.

Further, FIG. 25A illustrates examples of NAP recordings made with in-house made ‘self-holding’ sandwich electrodes from the ulnar nerve in the upper arm. The recording distance was 31 mm. A constant current stimulus (0.05 ms, 2 mA) was used. FIG. 25B illustrates a photograph and schematic drawings showing configurations of the electrodes and their actual placement. Both stimulation and recording electrodes contain three metal prongs with insulation except for the contact areas (indicated by arrowheads) and the grounding prong of the recording electrode. The nerve is placed between the middle prong and the outer 2 prongs of each electrode in a ‘sandwich’ manner so that the electrodes are held in place by the nerve alone in a hands- free manner without need for additional holding devices. The plastic base provides the electrode with some flexibility when holding the nerve.

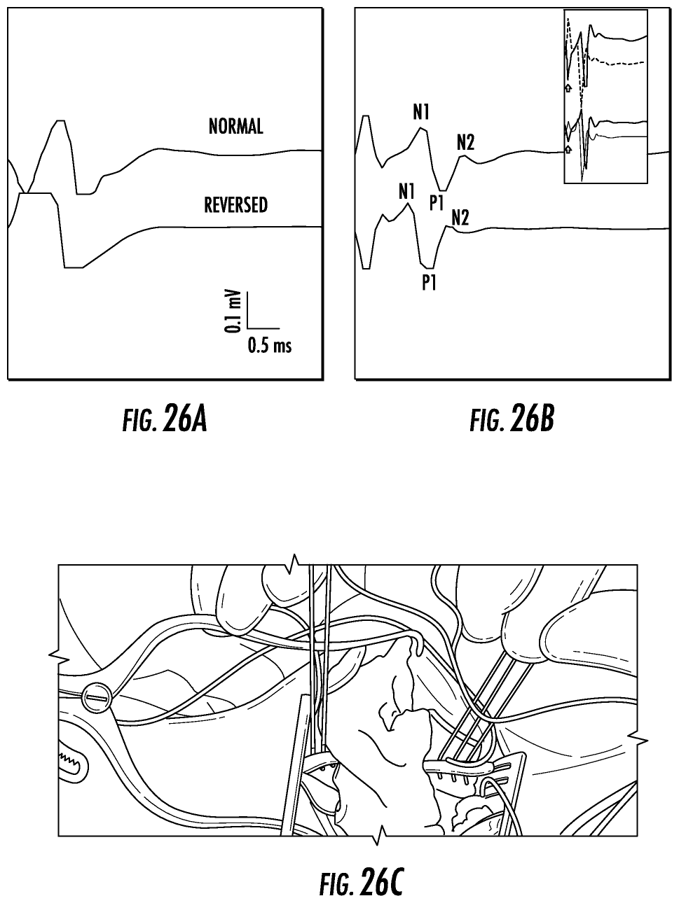

FIGS. 26A-26C illustrate graphical and image views of recording of NAPs with ‘bridge grounding’ in a clinical case, according to an embodiment of the present invention. The benefit of ‘bridge grounding’ was confirmed in an intraoperative recording in patients. In one example, the recordings were made from the medial antebrachial cutaneous nerve during brachial plexus exploration. Large stimulus artifacts were recorded when the nerve was lifted without ‘bridge grounding’ (FIG. 26A). After engaging ‘bridge grounding’, artifacts were suppressed and NAPs unmasked (FIG. 26B). NAP signals were confirmed by stimulus polarity switch- and intensity-response function tests. With ‘bridge grounding’, clean NAPs were recorded in both orthodromic and antidromic recordings. The recording arrangement is shown in FIG. 26C. The recording distance was 4.5 cm. It appears that NAPs recorded orthodromically are slightly larger than those recorded antidromically (Table 5). Conduction velocities did not differ between the two conduction directions. In addition, the NAP's amplitude saturated at a stimulus intensity of 2 mA. NAP latencies were consistently shorter for stimulation with reversed polarity than with normal polarity.

FIGS. 26A-26C illustrate ‘bridge grounding’ which unmasks NAPs recorded intraoperatively in a patient. NAPs were recorded with the standard IONM electrodes and an IONM machine. Large stimulus artifacts were recorded when the nerve was lifted and ‘bridge grounding’ was not implemented, as illustrated in FIG. 26A. After the ‘bridge grounding’ was placed and fully engaged, the artifacts were suppressed and NAP signals (N1, P1, and N2) emerged, as illustrated in FIG. 26B. The same stimuli (0.1 ms, 5 mA) were used in FIGS. 26A and 26B (the upper trace was from stimulation with normal polarity and the lower trace was from stimulation with reversed polarity in each panel). The intensity-response test was also performed in this case and the results are shown in an insert in FIG. 26B: 5 mA (two top traces) and 2 mA (two bottom traces). The stimulus artifacts are marked by arrows. In contrast to NAP signals, stimulus artifacts did not saturate in size with increasing intensity and their polarity changed with change in stimulus polarity. As illustrated in FIG. 26C, a photograph showing the recording arrangement. The medial antebrachial cutaneous nerve was dissected and isolated. A bipolar recording electrode and a tripolar stimulating electrode were held by hand and a large piece of saline-soaked gauze was placed between the recording and stimulation electrodes, serving as ‘bridge grounding’. A piece of insulation material was placed underneath the electrodes to isolate them from the surrounding tissue. The recording distance was 4.5 cm. Recordings were made at room temperature (18° C.).

TABLE 5
Measurements of NAPs recorded intraoperatively with ‘bridge grounding’
from the medial antebrachial cutaneous nerve in a patient.

| | Stimulus | | Peak | Peak | | |
|---|---|---|---|---|---|---|
| | Intensity | | amplitude (N1 | latency | Distance | CV |
| Recording | (mA) | Polarity | to P1, mV) | (N1, ms) | (mm) | (m/s) |
| Orthodromic | 2 | Normal | 0.19 | 1.0 | 45 | 45.0 |
| | | Reversed | 0.18 | 0.9 | 40 | 44.4 |
| | 5 | Normal | 0.17 | 1.0 | 45 | 45.0 |
| | | Reversed | 0.18 | 0.8 | 40 | 50.0 |
| Antidromic | 2 | Normal | 0.13 | 1.0 | 45 | 45.0 |
| | | Reversed | 0.14 | 0.8 | 40 | 50.0 |
| | 5 | Normal | 0.12 | 1.0 | 45 | 45.0 |
| | | Reversed | 0.14 | 0.8 | 40 | 50.0 |

CV, conduction velocity.

Previous publications recommend lifting the nerve out of the surgical field to achieve better intraoperative NAP recordings and to avoid current spread from the stimulating electrode to the tissue underneath. A recording distance of at least 4 cm was also recommended. In practice, however, recordings are often difficult to interpret due to large stimulus artifacts. Results from this study suggest that exaggerated stimulus artifacts may be caused by a loop effect created by lifting the nerve from the tissue underneath. In the model system, the artifact substantially reduced when the loop was cut open, indicating that it is as a major factor contributing to exaggerated stimulus artifact. The loop effect also explains why ‘bridge grounding’ in experiments and in clinical practice reduced the stimulus artifact in recordings from a lifted nerve. Minimizing the loop effect with the ‘non-lifting’ technique of the present invention also allowed for shorter recording distances of less than 4 cm.

This is believed to be the first study showing that the loop effect is the primary cause of a large stimulus artifact that often ruins NAP recordings from a surgically exposed nerve. The findings clearly imply that, contrary to what is typically recommended for NAP recordings, the nerve should not be lifted out of the surgical field and should stay in place (in situ) to have good contact with tissue underneath. However, good contact of the nerve segment tween the pairs of electrodes is difficult to achieve because the recording and stimulation electrodes have to be lifted away from tissue underneath. Furthermore, when there is not enough length of the nerve segment between the two pairs of electrodes, it may be impossible to allow the segment to touch the tissue underneath. Therefore, in this type of recording, the nerve segment is often entirely suspended in air and not touching the tissue underneath. In this case ‘bridge grounding’ needs to be applied.

Interestingly, an animal study previously reported that NAPs could only be recorded when the nerve between the recording and stimulating site was not dissected and isolated from the surrounding tissue. This configuration may be similar to the lifting nerve recordings with ‘bridge grounding’ and to clinical situations when the nerve between stimulation and recording electrodes are left in situ. In the same study, recordings were unsuccessful even when a nerve that did not touch the surrounding tissue was grounded between recording and stimulating site. This observation can be explained by the loop model because the electrical current can travel to the recording electrode via the loop formed by tissue underneath even when the direct pathway along the nerve is shunted by grounding.

The artifact issue has been discussed specifically with regard to the validity of intraoperative NAP recordings in brachial plexus lesion surgeries in infants. The infant plexus is small and relatively short. In a clinical report, Pondaag and colleagues concluded that intraoperative NAP recordings are not useful. They suggested a possibly different pathophysiology of the injured infant brachial plexus rather than suspecting technical challenges. However, as Kline and Happel pointed out, a representative NAP recording presented by Pondaag and colleagues seems to contain a large stimulus artifact outlasting the time within which, based on conduction time, a true NAP would have been expected. It is worth noting that Pondaag and colleagues reported that the nerve under study was fully dissected from the surrounding tissue and isolated from it with a dry surgical patty in their recordings. Kline and Happel successfully recorded NAPs from infants and argued that, although difficult, the recordings were useful for this type of surgery in infants. They did not provide details about how large artifacts were suppressed and neural signals were recognized. However, they suggested that improved instrumentation and technique should be developed for use in the pediatric population to allow application of the same decision-making principles used in the adult population. Avoiding the loop effect may help in the clinic with NAP monitoring in infants in whom the recording distance is short.

Change of stimulus polarity and an intensity-response function survey were used to identify neural signals and to differentiate neural signals from stimulus artifacts. This is the first report of NAP recognition with the aid of the stimulus polarity switch. It is easily performed (some IONM machines have a polarity switch button), time-efficient and produces a consistent result. It is recommended to use this routinely in intraoperative NAP recordings. Because the stimulation was performed with a tripolar electrode, the site of NAP generation changed slightly with polarity switching, and the signal latency always became shorter. The amount of latency shortening depended on the conduction velocity along the nerve and the inter-prong distance of the electrode. In addition, the intensity for maximal NAP increased slightly upon reversal of stimulus polarity reflecting a change in current density.

The loop hypothesis suggests that the ‘non-lifting’ configuration may be the best for intraoperative NAP recording. A new electrode for proof-of-concept of ‘non-lifting’ nerve recording in non-human primate was designed and tested.

The electrodes of the present invention are self-holding and self-insulated. Self-holding is made possible by the three-prong design. Three-pronged hook electrodes are commonly used for nerve stimulation in intraoperative NAP recording. Other types are also used, including straight and ball tip electrodes. NAP recordings are often made with bipolar electrodes. The use of a tripolar recording electrode was also reported with a ground-active-referential arrangement. However, the recordings for the present invention were differential.

When using non-insulated hook electrodes, the nerve needs to be lifted from the wet field potentially stretching the nerve and thereby affecting the recording. ‘Non-lifting’ nerve recording was reported using APS electrodes (the vagus nerve stimulation electrodes). In that report, only small NAP signals were shown following large stimulus artifacts in recordings made in monopolar stimulation and recording mode as bipolar mode was unsuccessful because of limited nerve length.

The results from tests with the new electrodes of the present invention in animals suggest that intraoperative NAP recordings may be substantially advanced by development of electrodes with self-insulation and self-holding features, equipped with multiple contact arrays and an electrical circuitry that allows automation of the stimulus polarity switch and intensity-response function tests.

Currently recommended clinical methods for intraoperative NAP recording originated from experiments in nonhuman primates. Exaggerated stimulus artifacts are identified as a major problem and found ‘bridge grounding’ to be a simple and effective solution. Ultimately, the new methodology of the present invention was brought forward into clinical practice where clinical rather than research equipment was used. The outcome was the same, validating the principle concept shared by recordings in these different settings.

The results of this study provide for the first time compelling, qualitative evidence that suggests that the loop effect may be the primary cause of an exaggerated stimulus artifact that often ruins NAP recordings from a short segment of surgically exposed peripheral nerves. This study was carried out using a limited number of animals and focused on qualitative descriptions of experimental findings. The study included a single clinical case where the ‘bridge grounding’ technique was applied for the first time in the OR to demonstrate and strengthen the significance of the findings related to the present invention. Since then, the technique was applied in two more cases and observed the same effect consistently. Nevertheless, for systematic and quantitative evaluations of these methods additional investigations in healthy and, more importantly, chronically injured nerves would be helpful and are needed.

Intraoperative nerve action potential (NAP) recordings are often affected by exaggerated stimulus artifacts. The results from experiments in nonhuman primate, a model system and clinical recordings, suggest that this technical challenge is embedded in the methodology where the exposed nerve is lifted from the surgical field, leading to a loop effect. This difficulty was resolved by application of ‘bridge grounding’ and configuration of ‘non-lifting’ nerve recording.

The proposed loop hypothesis and the reported solutions may significantly improve intraoperative NAP recording and clinical outcomes in various nerve repair surgeries.

In some embodiments, the present invention includes an electrode system for stimulation or recording of a compound nerve action potential having an elastic base or body with a minimum of two projections on one side and one on the opposing side of the nerve. The electrode placement would allow for “sandwiching” the electrode around the trunk of the nerve. The electrode is self-insulated and self-holding by allowing easy applications and a consistent and reliable ‘non-lifting’ nerve recording from surgically exposed peripheral nerve. The electrode includes a minimum of two metal contacts configured for an appropriate contact size, with none, one, or several on each projection.

The pressure on the nerve as the result of sandwiching it between the projections would apply enough pressure on the nerve to ensure sufficient and consistent electrical contact between the electrode contacts and the nerve. The pressure of the electrode on the nerve would apply less pressure than would be damaging to the nerve for the duration of the test. The projections comprise divots/grooves at the location of the electrode contact with an appropriate configuration and alignment to allow self-orienting positioning on the nerve once the electrode is positioned around the nerve.

The electrode is elastic and is formed in a way that would allow it to be open by squeezing either by fingers or with the use of common surgical tools (e.g. surgical tweezers) or special applicators to change the body of the electrode in a way that would fully open the electrode for positioning on the nerve. Once released from holding, the electrode would return to its original shape, fitting over the nerve with the self-orienting divots/grooves. The electrode would maintain its contact with the nerve in situ once it is released completely and positioned around the nerve. By squeezing the elastic body, the electrode can be opened in a way that would release pressure on the nerve completely allowing the electrode to be removed or repositioned.

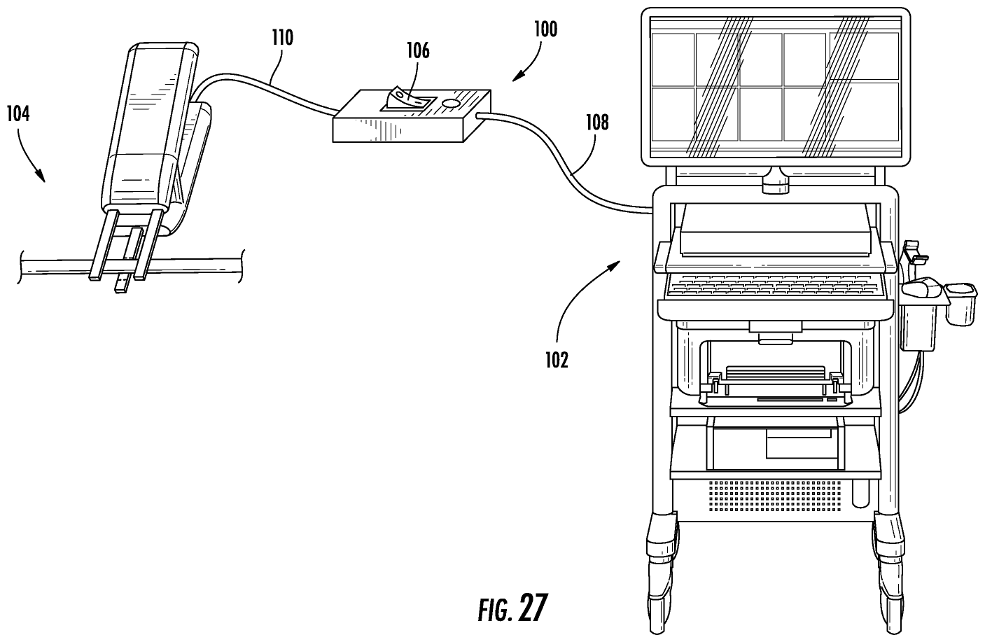

FIG. 27 illustrates a schematic view of an RTIM with a connected neural stimulator device and a GMG electrode, according to an embodiment of the present invention. In some embodiments of the present invention, a Real Time Impedance Monitor (RTIM) 100 plugs in on one side to the headboxes of the IONM machine or the Neural Stimulator (NS) 102 used to record from or stimulate the nerve. The GMG electrode 104 of the present invention plugs into the other side of the RTIM. The impedance monitor contains a switch 106 to allow the GMG electrodes to connect directly to the NS or the RTIM, as illustrated in FIG. 27. The devices are connected with wires 108, 110, as illustrated in FIG. 27, but could also be coupled with Bluetooth®, wifi, RFID, or any other means known to or conceivable to one of skill in the art.

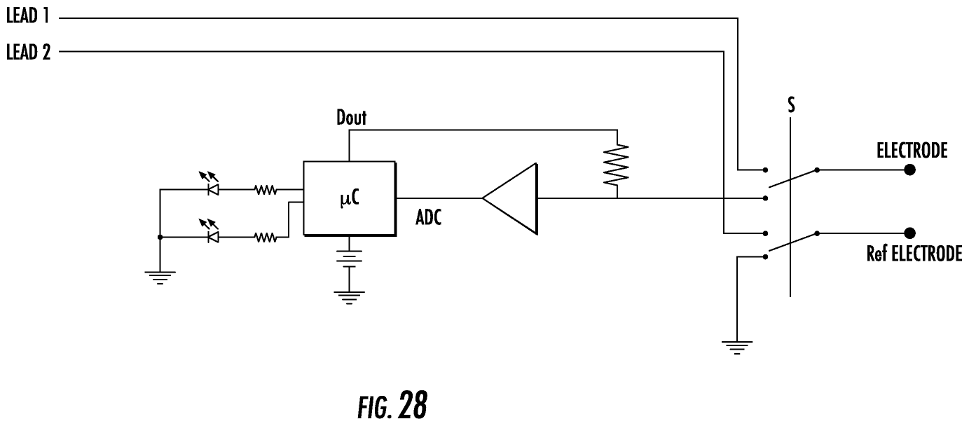

FIG. 28 illustrates a block diagram of the RTIM, according to an embodiment of the present invention. The RTIM device allows the surgeon to switch between using the device to determine the impedance and using the GMG electrode to stimulate/record from the nerve using the neural stimulator. The LEDs light red or green to indicate whether the electrode has a good connection to the nerve. When the switch S illustrated in FIG. 28 connects the electrodes to Leads 1 and 2 from NS, the GMG electrode can be used by the NS to stimulate or record without interference from RTIM.

Alternatively, the switch can connect the electrodes from GMG to RTIM. RTIM is a microcontroller-controlled device that delivers short voltage pulses via Dout, allowing instant impedance measurement of electrodes as a standalone device. The resulting current is delivered through the resistor to the Electrode. When the electrodes (Electrode and Ref Electrode) are connected across the tissue with the GMG electrode, the impedance will change. Low impedance corresponds to the electrodes shorted through body saline. High impedance corresponds to the electrodes not being connected. The impedance is measured as an output of the voltage divider via the op-amp. The output of the op-amp is sampled by the ADC of the microcontroller. Depending on the voltage output of the voltage divider that measures the impedance, the microcontroller will light the LEDs as red or green depending on the measured impedance. The LEDs are placed visible to the surgeon to provide visual aids to appropriate electrode placement.

The present invention carried out using a computer, non-transitory computer readable medium, or alternately a computing device or non-transitory computer readable medium incorporated into the NAP recording console. Indeed, any suitable method of calculation known to or conceivable by one of skill in the art could be used. It should also be noted that to the extent specific equations are detailed herein, variations on these equations can also be derived, and this application includes any such equation known to or conceivable by one of skill in the art.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can be a special computer designed specifically for this purpose. The computing device can be unique to the present invention and designed specifically to carry out the method of the present invention. It is not a standard business or personal computer that can be purchased at a local store. Additionally this computer carries out communications with the NAP recording device through the execution of proprietary custom built software that is designed and written by the scanner manufacturer for the computer hardware to specifically operate the scanner hardware.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A system for stimulation or recording of a compound nerve action potential comprising:
an elastic base, having at least three projections, wherein at least one of the at least three projections is positioned on a top side of a nerve and at least two of the at least three projections are positioned on a bottom side of the nerve, such that the nerve is sandwiched by the at least three projections;
an electrode for stimulation or recording of a nerve action potential comprising at least two metal contacts;
wherein the elastic base and the electrode are self-insulating and self-holding, such that a non-lifting nerve action potential can be stimulated and/or recorded.

2. The system of claim 1 wherein a middle projection and two outer projections comprise insulation that covers all of the middle projection and the two outer projections except for an area of the metal contact for nerve stimulation or recording.

3. The system of claim 1 further comprising the elastic base being formed from one selected from a group consisting of a plastic or silicone.

4. The system of claim 1 wherein a pressure is applied to the nerve because it is sandwiched by the at least three projections and wherein the pressure is less than what would be damaging to the nerve for a duration of a test of the compound nerve action potential.

5. The system of claim 1 wherein the at least three projections comprise grooves at a location of the electrode contact with the nerve, such that the nerve is self-positioned on the grooves.

6. The system of claim 1 wherein the projections can be opened by applying pressure to the elastic base using fingers or a surgical tool.

7. The system of claim 1 wherein the elastic base returns to its original shape after a deformation.

8. The system of claim 1 further comprising a device for applying deformation force to the elastic base.

9. The system of claim 1 wherein the elastic base is configured such that an application of deformation force to the elastic base releases pressure on the nerve.

10. The system of claim 1 wherein the elastic base is configured such that an application of deformation force allows the elastic base to be repositioned.

11. The system of claim 1 further comprising an impedance monitor.

12. The system of claim 11 wherein the impedance monitor measures impedance in real time.

13. The system of claim 11 wherein the impedance monitor couples the electrode to a neural stimulator device.

14. The system of claim 11 wherein the impedance monitor comprises a light emitting diode (LED).

15. The system of claim 14 wherein the LED is configured to indicate whether the electrode has a good connection to the nerve.

16. The system of claim 11 wherein the impedance monitor further comprises a switch, wherein the switch can be engaged to convert the electrode from stimulation and recording mode to impedance monitoring mode.

17. The system of claim 11 wherein the impedance monitor is configured to deliver short voltage pulses through the electrode.

18. The system of claim 11 wherein the impedance monitor further comprises an op-amp.

19. The system of claim 18 wherein the op-amp measures output from a voltage divider.

20. The system of claim 11 wherein a low impedance measurement indicates the electrode is shorted through saline in the body and a high impedance measurement indicates that the electrode is not connected.
